(19) United States
(12) Patent Application Publication
(10) Pub. No.: US 2026/0315399 A1
(43) Pub. Date: Oct. 15, 2026

(54) COATED INORGANIC SALTS AND USES THEREOF

(71) Applicant: Arbert, LLC, Spring House, PA (US)

(72) Inventors: Hemant N. Joshi, Parsippany, NJ (US); Craig Aronchick, Chico, CA (US); Gerald Bertiger, Spring House, PA (US)

(73) Assignee: Arbert, LLC, Spring House, PA (US)

(21) Appl. No.: 19/705,648

(22) Filed: Jun. 11, 2026

Related U.S. Application Data
(63) Continuation-in-part of application No. PCT/US24/59606, filed on Dec. 11, 2024.
(60) Provisional application No. 63/608,958, filed on Dec. 12, 2023.
(60) Provisional application No. 63/609,089, filed on Dec. 12, 2023.
(60) Provisional application No. 63/609,185, filed on Dec. 12, 2023.
(60) Provisional application No. 63/648,763, filed on May 17, 2024.
(60) Provisional application No. 63/676,209, filed on Jul. 26, 2024.

(51) Int. Cl. A61K 9/50; A61K 9/00; A61K 9/20; A61K 33/06; A61K 33/14; A61K 33/24; A61K 33/26; A61K 33/30; A61K 33/32; A61K 33/34; A61P 3/12

(52) U.S. Cl. CPC A61K 9/5047; A61K 9/0056; A61K 9/0095; A61K 9/2013; A61K 9/2054; A61K 9/2059; A61K 9/5015; A61K 9/5026; A61K 9/5031; A61K 9/5036; A61K 33/06; A61K 33/14; A61K 33/24; A61K 33/26; A61K 33/30; A61K 33/32; A61K 33/34; A61P 3/12

(57) ABSTRACT

Compositions of coated salts are disclosed. Also disclosed herein are methods of coating the inorganic salt(s), masking the taste of the salts and controlling the release of the salts in the gastro-intestinal tract. The granular salts can be coated as is. The powdered salts can be granulated first or spheronized or formed into minitablets or capsules prior to coating. The granulated coated salts are used in colonoscopy preparations and non-colonoscopy preparations.

### CROSS-REFERENCE TO RELATED APPLICATION

The present application is a Continuation-in-Part (CIP) of International Application No. PCT/US2024/059606, filed Dec. 11, 2024, which claims priority to U.S. Provisional Application No. 63/609,089, filed Dec. 12, 2023, to U.S. Provisional Application No. 63/608,958, filed Dec. 12, 2023, to U.S. Provisional Application No. 63/609,185, filed Dec. 12, 2023, to U.S. Provisional Application No. 63/648,763, filed May 17, 2024, and to U.S. Provisional Application No. 63/676,209, filed Jul. 26, 2024. The entire contents of each application are hereby incorporated by reference in its entirety.

### TECHNICAL FIELD

This patent document relates to coated salts. Also disclosed herein are methods of coating the inorganic salt(s), masking the taste of the salts and controlling the release of the salts. Also disclosed are compositions and kits of the salts for various indications.

### BACKGROUND

Sodium chloride and potassium chloride are key inorganic salts (i.e. electrolytes) in our body. Many other minerals such as calcium, zinc, copper, chromium, molybdenum, and manganese are also present in the human body. In many disease states, physical activities or as people age, there can occur an imbalance of electrolytes. For example, athletes, marathon runners, soldiers and other people involved in intense physical activity sweat significantly, thereby resulting in an electrolyte imbalance (e.g. hyponatremia and hypokalemia). Further, drinking a large volume of water in a short time can induce hyponatremia (dilutional hyponatremia). Adding a quantity of tasteless salts to free fluid will reduce the chances of dilutional hyponatremia during rehydration during exercise maintaining iso-osmolarity.

During colonoscopy, one has to take a preparation to cleanse the gastrointestinal tract with a large volume of water and during this process the patient can experience a loss of electrolytes. The preparation is a thickened liquid that is easy to swallow, especially for people suffering from dysphagia. The preparation uses ingredients which do not produce any insoluble matter in the GI tract, which can impede the colon examination. Similar side effects may be observed when people consume laxatives to treat chronic constipation, or have diseases causing nausea, vomiting, and/or diarrhea.

Therefore, it is important to maintain an appropriate level of each mineral (electrolyte) for appropriate functioning of tissues and organs. Although salt is consumed daily through food, it is very difficult to administer salt orally in large replacement quantities.

Acceptability of oral formulations depends upon four factors-appearance (eyes), odor (nose), taste (tongue) and texture to ensure patient compliance. Taste-masking is a more difficult task in many drug formulations. Addition of a flavor and a sweetener/sugar are normally and frequently tried first to mask the unpleasant taste of drugs. Taste receptors in the mouth are blocked sometimes to mask the unpleasant taste of the drug. Thresholds concentrations of primary taste sensation are—sweet (0.5%), sour (0.25%), salt (0.007%) and bitter (0.00005%). Clearly, one can taste even very small amounts of salts and bitter compounds.

Taste masking can be achieved by inclusion-complexes with cyclodextrins. The masking can also be achieved by ion-pairing the drug with ion-exchange resins. There are cationic and anionic resins, which are selected based on the charge on the drug molecule.

There is a need to develop methods to achieve taste masking of commonly used inorganic salt(s). However, it is also challenging to achieve a desired release rate of these salts in the GI tract.

### SUMMARY

Herein, the present disclosure provides a coating configuration for inorganic salt(s), which not only masks the taste of the salt but also provides a delayed release and/or sustained release in the GI tract. The present disclosure provides coated inorganic salt(s), compositions, kits and methods of preparation for a colonoscopy product that provides a delayed release and/or sustained release of electrolytes in the GI tract. Further, the present disclosure also provides balanced electrolyte compositions (i.e. sports drinks) comprising taste-masked electrolytes.

An aspect of the patent document provides a coated inorganic salt(s) comprising:
(a) one or more inorganic salt(s);
(b) a core comprising the one or more inorganic salt(s); and
(c) a first polymer coating layer enclosing the core; wherein the first polymer coating is an enteric or a reverse enteric coating, wherein the enteric coating dissolves in the small intestine and the reverse enteric coating dissolves in the stomach, wherein the concentration of salt ranges from about 5% to about 95% by weight in the composition.

In some embodiments, the one or more inorganic salt(s) is selected from the group consisting of sodium salt, potassium salt, calcium salt, iron salt, copper salt, zinc salt, manganese salt, magnesium salt, molybdenum salt, cobalt salt, and chromium salt, and/or the inorganic salt(s) comprise one or more anions selected from the group consisting of chloride, fluoride, sulfate, bisulfate, carbonate, bicarbonate, phosphate, citrate.

In some embodiments, the one or more inorganic salt(s) are admixed with a diluent and/or a gelling agent in the core, wherein the diluent or the gelling agent comprises one or more of polyethylene glycol, gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), corn starch, and carboxy methylcellulose, wherein the gelling agent is present in the core and swells upon contact with water thereby releasing the inorganic salt(s).

In some embodiments, the inorganic salt(s) is in the form of a plurality of granules in the core. In some embodiments, the granules have an average particle size smaller than 3000 microns.

In some embodiments, the coated inorganic salt(s) further comprise an undercoat between the core and the first polymer coating layer, wherein the undercoat prevents interaction between the inorganic salt(s) and the first polymer coating layer.

In some embodiments, the coated inorganic salt(s) further comprise a second polymer coating enclosing the core and the first polymer coating, wherein the second polymer coating delays the release of the inorganic salt(s), preferably wherein the second polymer comprises wax, PEG stearate coating, and any combination thereof.

In some embodiments, the core further comprises an inner core free from the inorganic salt(s), wherein the inorganic salt(s) are coated on the inner core. In some embodiments, the inner core consists essentially of one or more soluble materials, such that no insoluble residue remains after dissolution. In some embodiments, the one or more soluble materials are water-soluble sugar crystals. In some embodiments, the first polymer coating layer comprises hydroxypropyl methylcellulose phthalate (HPMC Phthalate), a methacrylic acid copolymer, or a combination thereof. In some embodiments, the coated inorganic salt further comprises a layer for providing extended release of the inorganic salt. In some embodiments, the coated inorganic salt is in the form of a bead.

In some embodiments, the coated inorganic salt(s) further comprise an extended-release layer between the core and the first polymer coating layer, wherein the coated inorganic salt(s) is configured so that, when tested with a USP type 2 dissolution system (Paddle Apparatus) at 50 rpm and a temperature of 37±0.5° C. in 125 mL 0.1 N HCl, at a pH of about 1.2 less than 20% of the salt is released within 90 minutes.

In some embodiments, the coated inorganic salt(s) further comprise an extended-release layer between the core and the first polymer coating layer, wherein the coated inorganic salt(s) is configured so that, tested with a USP type 2 dissolution system (Paddle Apparatus) at 50 rpm and a temperature of 37±0.5° C. in 125 mL of 0.05 M phosphate buffer, at a pH of about 6.8 or a pH of about 7.4,
(a) from about 0% to about 20% of the salt is released within 30 minutes;
(a) from about 30% to about 70% of the salt is released within 60 minutes; and
(b) from about 60% to about 100% of the salt is released within 90 minutes.

In some embodiments, the extended-release layer comprises one or more of ethyl cellulose, sodium polyacrylate, polyacrylamide copolymer, ethylene maleic anhydride copolymer, crosslinked carboxymethyl cellulose, polyvinyl alcohol copolymer, and cross-linked polyethylene oxide.

Another aspect provides a composition comprising the aforementioned coated inorganic salt.

Another aspect provides a dosage form comprising the aforementioned coated inorganic salt. In some embodiments, the dosage form is selected from the group consisting of beads, multiple particulates, tablets, capsules, caplets, sachets, granules, and liquid suspensions. In some embodiments, the dosage form is a hard or soft gelatin capsule. In some embodiments, the inorganic salt is in an effective amount to provide a normal electrolyte level. In some embodiments, the composition is in the form of a powder. In some embodiments, the composition is in the form of a tablet. In some embodiments, the composition further comprises a suspending agent, wherein the composition is in a liquid form. In some embodiments, the liquid form is a gel or a semi-solid.

Another aspect provides a kit comprising:
(a) the aforementioned coated inorganic salt(s), and
(b) a liquid medium.

In some embodiments, the liquid medium comprises a polyethylene glycol and/or water, wherein the polyethylene glycol is a colon cleansing agent. In some embodiments, the polyethylene glycol has a molecular weight ranging from 2000 daltons to 8000 daltons.

In some embodiments, the wherein the liquid medium comprises a suspending agent and/or water. Nonlimiting examples of suspending agents include xanthan gum, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof.

Another aspect provides a method of providing coated inorganic salt(s) to a subject in need thereof, the method comprising administering to the subject any of the aforementioned coated inorganic salt(s), compositions, or kits.

In some embodiments, the method provides balanced electrolytes. In some embodiments, the subject is electrolyte deficient. In some embodiments, the method reduces or masks the taste of the inorganic salt(s). In some embodiments, the coated inorganic salt(s) is delivered to a subject prior to a colonoscopy procedure.

### BRIEF DESCRIPTION OF THE DRAWINGS

FIG. 1 shows a pictorial diagram of a granule or a bead with various coatings, including (1) a coat (e.g. wax coating), (2) an enteric coating, (3) an coat (e.g. HPMC), (4) a diluent or gelling agent, and (5) the inorganic salt(s) (i.e. electrolytes). The core contains an electrolyte with a diluent and a gelling agent. The gelling agent provides a modified release of the electrolyte. The core is coated with a sub-coat, an enteric coat and a super-coat (wax coat).

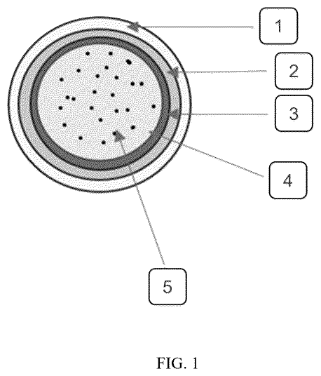

FIG. 2 is a pictorial diagram of the release of electrolyte from the core. Once the enteric coat dissolves (1), water is absorbed in the core (2). The gelling polymer swells slowing down the release of the inorganic salt(s) (3). Over time, the inorganic salt(s) diffuse out of the core (4).

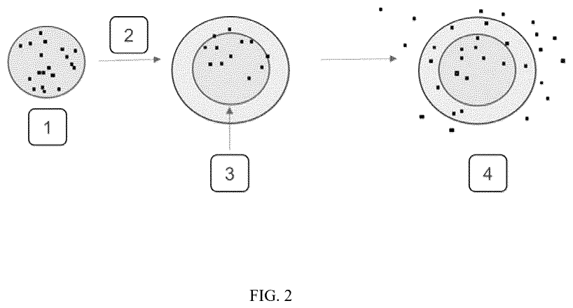

FIG. 3 is a pictorial diagram of particles for non-colonoscopy preparations. In this case, the core contains an electrolyte (6) with a diluent and a gelling agent (5). The core is coated with a sub-coat (4). There is a coat of water-insoluble, but water-swellable polymer, such as ethyl cellulose (1). This is further coated with an enteric coat (3). The last coat is a wax coat (2).

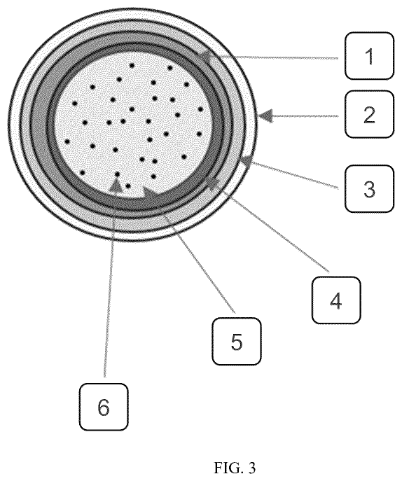

FIG. 4 is a pictorial diagram of a sugar bead or suglet (2) coated with electrolyte powder and gelling polymer (3). This is further coated with a sub-coat (1), enteric coat or a reverse enteric coat (4). The beads can be coated with water-insoluble but water swellable polymer and a wax coat.

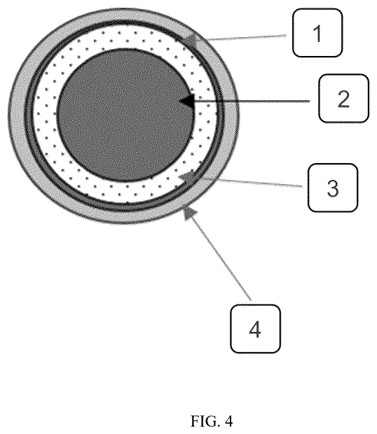

FIG. 5 is a depiction of various release profiles, such as immediate release (1), sustained release (2), delayed release followed by an extended/partial immediate release, (3) and delayed release followed by sustained release profile (4).

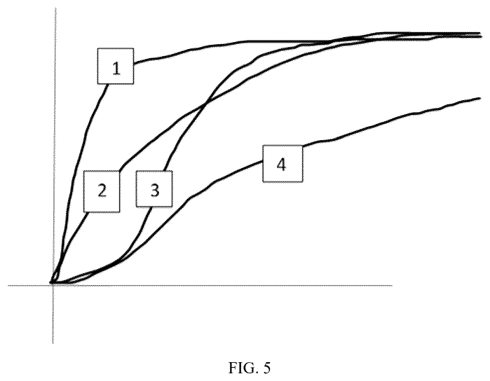

FIG. 6 is a table listing commercially available laxative MoviPrep, Plenvu, Gavilyte, Trilyte, CoLyte, Golytely, Nulytely, Halflytely, Suflave, and Suclear and respective ingredients.

### DETAILED DESCRIPTION

Various embodiments herein disclose a composition of a coated salt. In comparison with conventional salt forms, the coated salt disclosed herein can not only mask the taste but also provide the advantage of a delayed and/or extended release of the salt at a desired location for a suitable period of time. Meanwhile, incidences of adverse events such as nausea and vomiting can be eliminated or reduced. The coating approach can be adopted to various types of salts, in particular inorganic salt(s).

Although the following contents may refer to or exemplify a specific embodiment of a composition, they are not limited to the specified configuration or components of the composition. In view of practicality and economy considerations, a person skilled in the art can make various modifications to, e.g., the amounts of the salt and the excipients, and the form of the composition for suitable objectives.

### Definitions

Unless otherwise defined, all technical and scientific terms used herein have the same meaning as commonly understood by a person skilled in the field of the present invention. In case of conflict, the definitions provided in the application prevail.

The term “a”, “an” or “the” as used herein means “one or more” or “at least one”. That is, reference to any element or composition of the present invention by “a”, “an” or “the” does not exclude the possibility of the presence of a plurality of the elements and compositions.

The term “about” and the like as used herein, when used in connection with a numerical variable, generally means that the value of the variable and all values of the variable are within the range of experimental error (e.g., within the 95% confidence interval for the mean) or within ±10% or within ±5% of the indicated value.

The term “extended release” or “ER” as used herein refers to continuous release of a salt over a period of at least 30 minutes upon contact with a test medium or after being administered to the subject. In some embodiments, the period of continuous release is at least 1 hour, at least 2 hours, at least 3 hour, at least 4 hours, at least 6 hours, at least 8 hours, at least 10 hours, at least 15 hours, at least 20 hours, or at least 24 hours. Unless otherwise indicated, the amount of a salt released from the composition or dosage form is measured by dissolution testing in aqueous medium as described herein. The results of the dissolution testing are reported in terms of percentage content (w/w) released within the release time. The dissolution can be determined, for example, using a USP type 2 dissolution system (Paddle Apparatus) at 50 rpm and a temperature of 37±0.5° C. in a dissolution medium of 125 mL 0.1 N HCl. In some embodiments, the dissolution profile is determined using a USP type 2 dissolution system (Paddle Apparatus) at 50 rpm and a temperature of 37±0.5° C. in 125 mL of 0.05 M phosphate buffer, pH 6.8 or 7.4.

The term “immediate release” or “IR” as used herein refers to release of more than or equal to about 80% of a salt in less than or equal to about 30 minutes upon contact with a test medium or after being administered to the subject. In some embodiments, more than or equal to about 80% or more than or equal to about 90% or more than or equal to about 95% of the salt in an immediate release dosage form is released in less than or equal to about 30 minutes. Unless otherwise indicated, the amount of a salt released from the composition or dosage form is measured by dissolution testing in aqueous medium as described herein. The results of the dissolution testing are reported in terms of percentage content (w/w) released within the release time. The dissolution can be determined, for example, using a USP type 2 dissolution system (Paddle Apparatus) at 50 rpm and a temperature of 37±0.5° C. in a dissolution medium of 125 mL 0.1 N HCl. In some embodiments, the dissolution profile is determined using a USP type 2 dissolution system (Paddle Apparatus) at 50 rpm and a temperature of 37±0.5° C. in 125 mL of 0.05 M phosphate buffer, pH 6.8 or 7.4.

The term “coat” or “coating” refers a continuous and enclosing layer. The coating can serve various goals including masking the taste of a salt, controlling the release profile of the salt, and maintaining the quality or stability of the salt.

The term “functional coat” refers to a coating applied to an inorganic salt(s), which is designed to provide specific and desired release rate for the inorganic salt(s) including an immediate, delayed, extended-release properties and/or protective properties from various pH environments. Such coats include, but are not limited to, enteric coatings, reverse enteric coatings, and undercoating. The enteric coating functions to minimize the dissolution of inorganic salt(s) in the stomach and dissolve the inorganic salt(s) in the intestine. The reverse enteric coating functions to dissolve the inorganic salt(s) in the stomach, The undercoat functions to prevent interaction between the inorganic salts and the first polymer coating layer. Moreover, each coat serves a distinct function in achieving the desired delayed, extended release and/or protective properties.

As used herein, the terms “undercoat” and “seal coat” are used interchangeably and refer to a coating layer applied between the electrolyte-containing core and the enteric coating layer, which prevents interaction between the inorganic salt(s) and the enteric polymer.

The term “substantially free” as used herein with respect to a component (e.g., cornstarch) means that the component is present in an amount of less than about 1% by weight, preferably less than about 0.5% by weight, more preferably less than about 0.1% by weight, and most preferably is entirely absent. For example, a pure sugar bead that is “substantially free of cornstarch” contains less than about 1% cornstarch by weight of the bead, such that no visually detectable insoluble residue is produced upon complete dissolution of the bead in water.

The term “delayed release” means that the release of a salt does not initate immediately once ingested and the composition does not initiate the release of active until it reaches an environment of a predetermined pH range. Therefore, after being administered, the salt may not be released immediately and to reach the desired pH environment, it will take the salt composition a period of time, which may for example ranges from 5 minutes to 60 minutes, from 10 minutes to 60 minutes, or from 20 minutes to 60 minutes, or from 30 minutes to 60 minutes. The delayed release can prevent dumping of salts in the stomach.

The term “electrolyte” refers to a mineral that becomes ionized when dissolved in solvents like water, thus capable of conducting electricity. Unless otherwise specified, the terms salts and electrolytes can be used interchangeably. In some embodiments, the salt is an inorganic salt.

The term “coloring agent” refers to an agent generates a desired color.

The term “flavors” refers to an agent, which when added to the dosage form, provides a pleasing smell or odor.

The term “sweeteners” refers to an agent, which when added to the dosage form, incorporates a desired sweetness.

The term “taste enhancing agent” refers to an agent, which stimulates taste sensation and provides a pleasing taste in the mouth cavity.

The term “subject” as used herein is intended to include human and non-human animals. Non-human animals include all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. In some embodiments, the subject is a colonoscopy patient. In some embodiments, the colonoscopy patient suffers from dysphagia. In another embodiment, the subject has a lower-than-normal level of electrolytes. In some embodiments, the subject is a person who is electrolyte deficient as a result of physical exercise. In some embodiments, the subject is an athlete or a sportsperson.

The term “electrolyte deficient” refers to the subject lacking one or more inorganic salt(s), such as sodium chloride, potassium chloride, calcium chloride, or other inorganic salt(s) commonly used in electrolyte formulations. In some embodiment, electrolyte deficient means the subject has a lower-than-normal level of electrolytes.

A “normal level” of electrolytes is an average level of electrolytes measured from a group of healthy individuals. A lower-than-normal level of electrolytes (i.e., an electrolyte imbalance) can result from a decrease in electrolytes of least 1%, 5%, at least 10%, or at least 15% in electrolytes compared to a normal level.

The term “configured” means that the coated inorganic salt, or compositions thereof, have been intentionally designed to achieve a specific function. For example, the coated inorganic salt(s) have been deliberately designed to produce particular release profiles (extended and delayed release), controlling release timing, and/or masking the taste of the inorganic salt(s).

The term “dosage form” as used herein refers to a physical form in which the coated inorganic salt or composition thereof is presented for administration to a subject. Nonlimiting examples of dosage forms include beads, multiple particulates, tablets, capsules, caplets, sachets, granules, liquid suspensions, gels, powders, and beverage mixes. The dosage form may comprise a single coated inorganic salt or a plurality of coated inorganic salts. In some embodiments, the dosage form is a hard or soft gelatin capsule containing the coated inorganic salt. In some embodiments, the dosage form is a sachet containing a dry powder or granule form of the coated inorganic salt, which may be reconstituted with water or another liquid prior to administration. In some embodiments, the dosage form is a beverage mix comprising the coated inorganic salt along with one or more of a sweetener, a flavoring agent, a gelling agent, and a diluent, which is intended to be dissolved or dispersed in water or another potable liquid to form a drinkable preparation.

### 1. Coated Inorganic Salt(s)

An aspect of the patent document provides a coated inorganic salt comprising (a) one or more inorganic salt; (b) a core comprising the one or more inorganic salt; and (c) a first polymer coating layer enclosing the core; wherein the concentration of salt ranges from about 5% to about 95% by weight in the composition.

Various inorganic salt(s) can be incorporated into the coated inorganic salt. Nonlimiting examples include sodium salt (e.g. sodium chloride, sodium bicarbonate, sodium carbonate, sodium sulfate, sodium phosphate), potassium salt (e.g. potassium chloride, potassium carbonate, potassium bicarbonate, potassium sulfate, potassium phosphate), calcium salt, iron salt, copper salt, zinc salt, manganese salt, magnesium salt, molybdenum salt, cobalt salt, chromium salt, salts of halide (F, Br, Cl, or I). The amount of the salt in the coated inorganic salt ranges from about 10% to about 95%, from about 20% to about 80%, from about 30% to about 95%, from about 40% to about 80%, or from about 40% to about 60% by weight in the coated inorganic salt. Nonlimiting examples of the amount include about 10%, about 15%, about 20%, about 25%, about 30%, about 35%, about 40%, about 45%, about 50%, about 55%, about 60%, about 65%, about 70%, about 80%, about 90%, about 95%, and any range between any two of the aforementioned values. In some embodiments, the coated inorganic salt is free from other ingredients for therapeutic purposes.

A mixture of salts can be coated using the same methods disclosed herein. Benefit of coating a mixture includes saving of time of coating, saving of cost, ease of handling materials during packaging etc. The mixture may include for example sodium chloride+potassium chloride, sodium chloride+sodium sulfate, sodium chloride+sodium bicarbonate, sodium chloride+potassium chloride+sodium sulfate, sodium chloride+potassium chloride+sodium sulfate+sodium bicarbonate, etc. The salt mixture can be in the form of particles or granules prior to coating. In some embodiments, the active agent that elicits any therapeutic or physiological effect in the coated inorganic salt consists essentially of the inorganic salt.

The polymer for the first polymer coating layer can be selected for any suitable purpose including for example masking the taste of the salt, delaying the release of the salt, and enabling an extended release of the salt. In some embodiments, the amount of the first polymer ranges from about 0.1% to about 50%, from about 0.1% to about 25%, from about 2% to about 40%, from about 1% to about 10%, from about 5% to about 10%, or from about 10% to about 20% in the coated inorganic salt. Nonlimiting examples of the amount include about 0.1%, about 0.5%, about 1%, about 2%, about 4%, about 6%, about 8%, about 10%, about 20%, about 25%, about 30%, about 40%, about 50%, and any range between any two of the aforementioned values.

In some embodiments, the first polymer coating layer serves a role of an enteric coat. In some embodiments, this coating does not dissolve in acidic pHs, for example below pH 4, below pH 4.5 or below pH 5. Nonlimiting examples of polymers for such a purpose include cellulose acetate phthalate (CAP), cellulose acetate trimellitate (CAT), Hydroxypropyl methylcellulose phthalate (HPMCP), hydroxypropyl methylcellulose acetate succinate (HPMCAS), polyvinyl acetate phthalate (PVAP), methacrylic acid copolymers (Eudragit L30 D 55, L100 D 55, L100, S 100, S12.5, L 12.5, FS 30D and Shellac and any combination thereof. Methyacrylate polymers may be of various types and grades including for example methyl acrylate, methyl methacrylate and methacrylic acid (7:3:1) copolymer, methacrylic acid-ethyl acrylate (1:1) copolymer, methacrylic acid-methyl methacrylate (1:1) copolymer, methacrylic acid-methyl methacrylate (1:2) copolymer etc. Methacrylic acid-methyl methacrylate (1:2) copolymer can be used to target the release above pH 7. Methacrylic acid-ethyl acrylate (1:1) copolymer can be used to target the release above pH 5.5. Mixtures of these polymers can also be used to control the release of the salt at certain pH values.

In some embodiments, the first polymer coating layer comprises hydroxypropyl methylcellulose phthalate (HPMC Phthalate, also known as hypromellose phthalate). HPMC Phthalate provides certain advantages over methacrylic acid copolymers (Eudragit) when used in formulations containing high concentrations of electrolytes. In some embodiments, methacrylic acid copolymers (including Eudragit L100-55 and Eudragit L30D-55) form a lumpy mass in the presence of high amounts of electrolytes, which may decrease the release of salts. HPMC Phthalate avoids this lumpy mass formation while providing equivalent or superior enteric protection. In some embodiments, HPMC Phthalate requires a lower percentage of coating compared to methacrylic acid copolymers to achieve comparable acid resistance. For example, about 15% HPMC Phthalate coating provides equivalent protection to about 20% Eudragit coating for Golytely-type formulations. In some embodiments, as little as about 5% HPMC Phthalate achieves excellent acid resistance with less than about 2.07% release at pH 1.2. In some embodiments, the HPMC Phthalate coating ranges from about 3% to about 20%, from about 5% to about 15%, or from about 5% to about 12% by weight of the coated inorganic salt.

In some embodiments, the first polymer coating layer serves a role of a reverse enteric coat. In some embodiments, this coating does not dissolve in neutral/basic pH, for example, above pH 4.5, above pH 5, above pH 5.5 or above pH 6. Nonlimiting examples of suitable polymers include basic butylated methacrylate copolymer, amino methacrylate copolymer, and aminoalkyl methacrylate copolymer E.

The first polymer coating layer may contain one or both of the enteric coat and the reverse enteric coat. In some embodiments, the core is coated with a reverse enteric coat followed by an enteric coat. In some embodiments, the core is coated with a mixture of enteric coat polymer(s) and reverse enteric coat polymer(s) to obtain a desire dissolution profile in the stomach.

Besides the inorganic salt, the core may also contain an excipient such as a diluent and a gelling agent admixed with the salt. In some embodiments, the gelling agent can swell upon contact with water thereby releasing over a period of time the inorganic salt from the porosity resulting from gel swelling. Nonlimiting examples of the diluent or filler materials include lactose, sucrose, sorbitol, mannitol, and xylitol. Nonlimiting examples of the gelling agent include hydroxypropyl methyl cellulose (HPMC), methyl cellulose (MC), hydroxy propyl cellulose (HPC), carboxy methyl cellulose (CMC), sodium carboxymethyl cellulose, xanthan gum, gellan gum, alginic acid, sodium alginate, hyaluronic acid, sodium hyaluronate, povidone, and Chitosan. Some agents can play the role of both a diluent and a gelling agent. The amount of the diluent or gelling agent may vary depending on the specific salt and the rate of delayed or controlled release. In some embodiments, the amount of the diluent or gelling agent independently ranges from about 2% to about 80%, from about 5% to about 70%, from about 10% to about 60%, from about 20% to about 50%, or from about 20% to about 40% in the coated inorganic salt. Nonlimiting examples of the amount include about 5%, about 10%, about 15%, about 20%, about 25%, about 30%, about 40%, about 50%, about 60%, and any range between any two of the aforementioned values.

The core may also contain an inner core, where the salt is coated on the inner core. Preferably, the inner core is inert and free from the inorganic salt. For instance, a mixture of powdered salt and a sustained release polymer can be coated on sugar beads or suglets or glucose beads, which can be further coated with one or more layers of functional coatings. In some embodiments, the D90 value (it is the value of the particle size below which 90% of particles reside) of the uncoated suglets particles is less than 700 μm. D90 of less than 700 μm means 90% of the particles are less than 700 μm.

The core may also contain an inner core, where the salt is coated on the inner core. Preferably, the inner core is inert and free from the inorganic salt(s). In some embodiments, the inner core consists essentially of one or more soluble materials, such that no insoluble residue remains upon dissolution. In some embodiments, the one or more soluble materials are water-soluble sugar crystals. In some embodiments, the water-soluble sugar crystals are selected from the group consisting of maltose, sucrose, glucose, fructose, lactose, and combinations thereof. For instance, a mixture of powdered salt and a sustained release polymer can be coated on sugar beads or suglets or glucose beads, which can be further coated with one or more layers of functional coatings. In some embodiments, the D90 value (the particle size below which 90% of particles reside) of the uncoated suglets particles is less than 700 μm.

In a preferred embodiment for colonoscopy applications, the inner core comprises a pure sugar bead that is substantially free of cornstarch. Commercially available sugar beads (suglets) typically contain cornstarch as a structural component. However, cornstarch is not water-soluble and produces opaque, insoluble particles that can obscure colonic mucosa during colonoscopy examination, potentially masking polyps and significantly decreasing the sensitivity and specificity of the procedure as a screening technique to prevent or detect early colon cancer. Therefore, the present disclosure provides a pure sugar bead inner core consisting essentially of one or more sugars, prepared by coating sugar powder onto sugar crystals using sugar syrup as the binding agent. When water from the sugar syrup evaporates during manufacturing, it produces an additional sugar coating layer, thereby building up uniform, spherical, entirely water-soluble beads free of cornstarch or other water-insoluble components.

In some embodiments, the pure sugar bead comprises: (a) water-soluble sugar crystals as the seed core, present in an amount of about 1% to about 99% by weight of the bead, including about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range between any two of the aforementioned values; (b) sugar powder coated onto the sugar crystals, present in an amount of about 1% to about 99% by weight of the bead, including about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the bead, and any range between any two of the aforementioned values; and (c) sugar syrup as the binder, wherein evaporation of water from the sugar syrup during the coating process produces an additional sugar coating layer. In some embodiments, the sugar is sucrose. In some embodiments, the sugar is selected from the group consisting of maltose, sucrose, glucose, fructose, lactose, and any combination thereof. In some embodiments, the pure sugar bead core is 100% sucrose. In some embodiments, the sugar is selected from one that metabolizes to glucose.

In some embodiments, the starting sugar crystals have a particle size of about 10 mesh to about 15 mesh (approximately 1.0 mm to 2.0 mm). In some embodiments, the sugar powder has a particle size of about 30 mesh to about 50 mesh. In some embodiments, the finished pure sugar bead has a diameter of up to about 1 mm. Other particle sizes are acceptable and the starting crystal size may be selected based on the desired final bead diameter and downstream coating requirements. In some embodiments, the pure sugar bead is free of any water-insoluble excipients, such that when the bead and all subsequent coatings dissolve in the gastrointestinal tract, no insoluble residue remains that could interfere with endoscopic visualization.

The salt in the coated inorganic salt may exist in its natural form such as granules or crystals. The salt may also be granulated using a binding agent before being coated or admixed with an excipient. For example, the salt to be coated can be first granulated with excipients to form granules or beads or microspheres, which optionally provide an additional level of control over sustained release of salts in the GI tract. In further examples, the salt is first granulated with a polymer to provide an extended or sustained release profile because of the swelling of polymer, and the granules are then enteric-coated or reverse enteric coated and/or wax coated. Of course, the binding agent may also play little or no role in extended release depending on the selection of the agent and its amount. Nonlimiting examples of the binding agent include HPMC, HPC, lactose, mannitol, sorbitol, xanthan gum, Sodium CMC, and any combination thereof.

The particle size of electrolytes or salts is very important. Sodium chloride is available in the granular form. Potassium chloride is available in the granular form, but the particle size is smaller than the granules of sodium chloride. Sodium bicarbonate is available mostly as a fine powder. It is available as granules, but the particle size is significantly smaller than sodium chloride granules. Sodium sulfate and magnesium sulfate are available as fine powders. Once granulated with a diluent and/or a gelling agent, the particle size of granules should not be too large and the patient should not feel the grittiness of the electrolyte granules.

In some embodiments, the binding agent and its amount are selected so that the granules have an average particle size (or D90) ranging from about 20 to about 5000 microns, from about 20 to about 5000 microns, from about 100 to about 3000 microns, from about 20 to about 2000 microns, from about 50 to about 1500 microns, from about 100 to about 1500 microns, from about 200 to about 1000 microns, or from about 500 to about 1000 microns. Nonlimiting examples of the average size include about 20 microns, about 50 microns, about 100 microns, about 150 microns, about 200 microns, about 250 microns, about 300 microns, about 300 microns, about 400 microns, about 500 microns, about 600 microns, about 800 microns, about 1000 microns, about 1200 microns, about 1500 microns, about 2000 microns, about 2500 microns, about 3000 microns, about 3500 microns, about 4000 microns, about 5000 microns, about 6000 microns, about 8000 microns, and any range between any two of the aforementioned values.

In some embodiments, the coated inorganic salt further comprises a layer for providing extended release of the inorganic salt. The core may be further coated with an extended or modified release layer between the core and the first polymer coating layer. This layer is different from the undercoat. In some embodiments for example, the salt is coated with water-insoluble, but water-swellable polymer such as ethyl cellulose to obtain a modified release of the salt. The coating can be a mixture of water-insoluble/water-swellable polymer and water-soluble polymer to alter the porosity of the coating. The extended or modified release coat ranges from about 2% to about 80%, from about 5% to about 70%, from about 10% to about 60%, from about 2% to about 40%, from about 50% to about 30%, or from about 10% to about 20% by weight in the coated inorganic salt. Nonlimiting examples of the amount include about 2%, about 5%, about 8%, about 10%, about 15%, about 20%, about 25%, about 30%, about 40%, about 50%, about 60%, and any range between any two of the aforementioned values.

Nonlimiting examples of the excipient in the extended-release layer include ethyl cellulose, sodium polyacrylate, polyacrylamide copolymer, ethylene maleic anhydride copolymer, crosslinked carboxymethyl cellulose, polyvinyl alcohol copolymer, cross-linked polyethylene oxide, and any combination thereof.

In some embodiments, the coated inorganic salt further includes an undercoat between the core and the first polymer coating layer. This undercoat can prevent interaction between the electrolyte and the polymer used for enteric coating. Preferably, the undercoat is soluble in water. Nonlimiting examples of excipients suitable for the undercoat include methyl cellulose, hydroxypropyl methylcellulose, carboxymethyl cellulose, and polyvinyl alcohol. In some embodiments, the undercoat does not delay or extend the release but instead serves as an immediate release layer. In some embodiments, the salt core is coated with an immediate release polymer (undercoat) followed by an enteric coating. In some embodiments, the salt core is coated with an immediate release polymer (undercoat) followed by a wax coating.

The undercoat may range from about 0.1% to about 20%, from about 0.1% to about 15%, from about 0.1% to about 10%, from about 1% to about 10%, from about 5% to about 10%, or from about 10% to about 20% in the coated inorganic salt. Nonlimiting examples of the amount include about 0.1%, about 0.5%, about 1%, about 2%, about 4%, about 6%, about 8%, about 10%, and any range between any two of the aforementioned values.

In some embodiments, the coated inorganic salt further includes a second polymer coating enclosing the core and the first polymer coating. In some embodiments, the second polymer coating dissolves slowly in water delaying/slowing the release of electrolytes. Nonlimiting examples of the second polymer include wax, PEG stearate, carnauba wax, bees wax, shellac, a high molecular weight polymer or combination thereof.

In some embodiments, each coating of the coated inorganic salt is water-soluble. For example, in the colonoscopy preparation, all the ingredients in the coated inorganic salt or dosage form must be water-soluble so that none of the colonic mucosa is obscured by the insoluble residue. In some embodiments, one or more coating of the coated inorganic salt is water insoluble. In some embodiments for the non-colonoscopy preparation, some of the ingredients for coatings may not be water-soluble.

Other excipients can also be included in the coated inorganic salt disclosed herein, including for example plasticizer, flavor, taste enhancer, stabilizer. Nonlimiting examples of plasticizers include diethyl phthalate, tributyl citrate, triethyl citrate, glycerin, propylene glycol, polyethylene glycol, tributyrin. For instance, PEG 3350 at 10% of the enteric coating polymer level can be used. A solvent mixture containing ethanol, acetone and water at 50%, 20% and 30%, respectively can be used to facilitate the process. In some embodiments, other ratios of these solvents are used. In some embodiments, other solvents, such as isopropyl alcohol, can be used in the solvent mixture.

Delayed/extended release

In some embodiments, the coated inorganic salt is configured, and the excipients or polymers and their amounts are selected so that the inorganic salt of the coated inorganic salt is controlled via delayed release, immediate release, and/or extended release in an environment of a predetermined pH or a predetermined pH range. For example, a super coated (e.g. wax coating) particle or tablet dissolves slowly in intestine thereby delaying the release of the salt. Meanwhile, an inner extended-release layer (e.g., ethyl cellulose coating), after the dissolution of the super-coat, will swell in the intestine thereby releasing the salt in an extended-release fashion. In some embodiments, the coating layers (e.g. super-coat, first polymer coating, and/or second polymer coating) of the coated inorganic salt are configured to dissolve only under a predetermined pH, including for example, 2, 2.5, 3.0, 3.5, 4.0, 4.5, 5.0, 5.5, 6.0, 6.5, 7.0, 7.5, 8.0, 8.5, 9.0, 9.5, or 10.0, or at a range between any two of the aforementioned values.

In some embodiments, it is important to keep the Cmax value (maximum salt concentration achieved in the blood when the salt is administered in a dosage form) low during the absorption of salts. Therefore, the salts should be released slowly from the coated inorganic salt (sustained or slow-release system or dosage form) but the AUC (area under the drug absorption curve) needs to be satisfactory.

In some embodiments, the coated inorganic salt is configured, and the excipients or polymers and their amounts are selected so that the inorganic salt of the coated inorganic salt has one or more or all of the following in vitro dissolution characteristics in the dissolution media having pH of about 6.5 to 7.5.
(a) in 0.25 hour, the released salt accounts for about 0.01% to about 5%, about 0.01% to about 4%, about 0.01% to about 3%, about 0.01% to about 2%, about 0.01% to about 1%, about 0.01% to about 0.1%, about 0.01% to about 0.05%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1% of the total amount of the salt (w/w);
(b) in 0.5 hour, the released salt accounts for about 0.1% to about 20%, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 6%, about 0.1% to about 4%, about 0.1% to about 3%, about 0.1% to about 2%, about 0.1% to about 1%, about 0.5% to about 10%, about 0.5% to about 8%, about 0.5% to about 6%, about 0.5% to about 6%, about 0.5% to about 4%, about 0.5% to about 3%, about 0.5% to about 2%, about 0.5% to about 1%, about 1% to about 10%, about 1% to about 8%, about 1% to about 6%, about 1% to about 6%, about 1% to about 4%, about 1% to about 3%, about 1% to about 2%, about 1% to about 1% of the total amount of the salt (w/w);
(c) in 1 hours, the released salt accounts for about 2% to about 30%, about 2% to about 25%, about 2% to about 20%, about 2% to about 15%, about 2% to about 10%, about 2% to about 8%, about 5% to about 30%, about 5% to about 25%, about 5% to about 20%, about 5% to about 15%, about 5% to about 10%, about 8% to about 30%, about 8% to about 25%, about 8% to about 20%, about 8% to about 15%, or about 8% to about 12% of the total amount (w/w);
(d) in 2 hours, the released salt accounts for about 2% to about 40%, about 2% to about 30%, about 2% to about 25%, about 2% to about 20%, about 2% to about 15%, about 2% to about 10%, about 2% to about 8%, about 5% to about 40%, about 5% to about 30%, about 5% to about 25%, about 5% to about 20%, about 5% to about 15%, about 5% to about 10%, about 8% to about 40%, about 8% to about 30%, about 8% to about 25%, about 8% to about 20%, about 8% to about 15%, or about 8% to about 12% of the total amount (w/w); and/or
(e) in 4 hours, 5 hours, 6 hours, 7 hours, 8 hours, 10 hours or 15 hours, the released salt accounts for about 50% to about 100%, about 70% to about 100%, about 60% to about 100%, about 50% to about 95%, about 70% to about 95%, or about 80% to about 100% of the total amount (w/w). In some embodiments, the coated inorganic salt has one, two, three or four of the above dissolution characteristics. In some embodiments, the coated inorganic salt provides the dissolution of the active ingredient having the above (a). In some embodiments, the coated inorganic salt provides the dissolution of the active ingredient having the above (a) and (b). In some embodiments, the coated inorganic salt provides the dissolution of the salt having the above (a), (b) and (c). In some embodiments, the coated inorganic salt provides the dissolution of the salt having the above (a), (b), (c) and (d). In some embodiments, the coated inorganic salt provides the dissolution of the salt having the above (a), (b), (c), (d), and (e).

In some embodiments, the coated inorganic salt is configured to provide a delayed-release profile wherein no more than 25%, and preferably, no more than 10% of electrolytes dissolve in stomach. Further, in some embodiments, the coated inorganic salt is configured to provide an extended release in the intestine over a time frame of about 30 minutes to about 60 minutes.

In some embodiments, the coated inorganic salt has the following in-vitro dissolution for the release of the salt: less than 40% is released in 2 hours, preferably less than 30% is released within 2 hours, more preferably less than 30% is released in 2 hours, more preferably less than 20% is released in 2 hours, more preferably, less than 10% is released in 2 hours, most preferably no salt is released in 2 hours.

In some embodiments, the coated inorganic salt has the following in-vitro dissolution for the release of the salt in pH 1.2: less than 40%, preferably less than 30%, more preferably less than 20%, more preferably, less than 9%, more preferably, less than 8%, more preferably, less than 7%, more preferably, less than 6%, more preferably, less than 5%, more preferably, less than 4%, more preferably, less than 3%, more preferably, less than 2%, more preferably, less than 1%, more preferably, less than 0.1% is released, and most preferably no salt is released within 30 minutes, within 60 minutes, within 90 minutes, or within 120 minutes.

In some typical embodiments, the coated inorganic salt has the following in-vitro dissolution for the release of the salt in pH 7.4: (a) the released salt is about 0%-40%, about 5%-35%, about 10%-30%, about 15%-25% or about 20% (e.g. about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about, 21%, about 22%, about 23%, about 24%, about 25%, about 26%, about 27%, about 28%, about 29%, or about 30%, about 31%, about 32%, about 33%, about 34%, about 35%, about 36%, about 37%, about 38%, about 39%, or about 40%) of the total amount of the salt in 0.5 hour, (b) the released salt is about 30%-70%, about 40%-60%, about 45%-55% or about 50% (e.g. about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about, 51%, about 52%, about 53%, about 54%, about 55%, about 56%, about 57%, about 58%, about 59%, or about 60%, about 61%, about 62%, about 63%, about 64%, about 65%, about 66%, about 67%, about 68%, about 69%, or about 70%) in 1 hour, and/or (c) the released salt is about 60%-100%, about 70%-90%, about 80%-90%, or about 85% (e.g. about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about, 81%, about 82%, about 83%, about 84%, about 85%, about 86%, about 87%, about 88%, about 89%, or about 90%, about 91%, about 92%, about 93%, about 94%, about 95%, about 96%, about 97%, about 98%, about 99%, or about 100%) in 1.5 hours. In some embodiments, the salt is released according to the above (a), (a)+(b), (a)+(c), (b)+(c), or (a)+(b)+(c).

The above dissolution can be determined using USP Apparatus II (paddle). Specific procedures are illustrated in the examples. In some embodiments, the dissolution profile is determined using a USP type 2 dissolution system (Paddle Apparatus) at 50 rpm and a temperature of 37±0.5° C. in 125 mL 0.1 N HCl for 60 minutes. In some embodiments, the dissolution profile is determined using a USP type 2 dissolution system (Paddle Apparatus) at 100 rpm and a temperature of 37±0.5° C. in 900 mL 0.1 N HCl for 60 minutes. This mimics the dissolution of electrolytes from the dosage form (beads, granules etc.) in the stomach. In order to mimic the dissolution of electrolytes from the dosage form in the intestine, a pH 7.4 buffer was used. In some embodiments, the dissolution profile is determined using a USP type 2 dissolution system (Paddle Apparatus) at 50 rpm and a temperature of 37±0.5° C. in 125 mL of 0.05 M phosphate buffer, pH 6.8 or 7.4 for 60 minutes. In some embodiments, the dissolution profile is determined using a USP type 2 dissolution system (Paddle Apparatus) at 100 rpm and a temperature of 37±0.5° C. in 900 mL of 0.05 M phosphate buffer, pH 6.8 or pH 7.4 for 60 minutes. The sampling time points could be 10 min, 20 min, 30 min, 45 min and 60 min.

In some embodiments, the coated inorganic salt is configured to provide an extended release over a period of 30 minute, 45 minutes, 1 hour, 2 hours, 4 hours, 6 hours, 8 hours, 10 hours, 15 hours, 20 hours, 24 hours, or any range between any two of the aforementioned values. The extended release may take place in stomach, small intestine and/or large intestine.

### 2. Compositions of Coated Inorganic Salt(s)

An aspect of the patent document provides a composition comprises the above disclosed coated inorganic salt. In some embodiments, the compositions may be used for colonoscopy preparations, sports drinks and/or any situation which necessitates the replenishment of electrolytes. In some embodiments, the compositions may be configured to have to same delayed release profile as disclosed above.

The composition may be in any suitable form. In some embodiments, the composition may be in the form of a powder, a granule, tablets, capsules, a particle, a gel, or a suspension. In a preferred embodiment, the composition is in the form of a liquid, further comprising a suspending agent selected from, but not limited to xanthan gum, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof, wherein the polymer-coated core is suspended in the liquid.

In some embodiments, the composition or dosage form is in the form of tablets, minitablets, beads, multiple particulates, capsules, caplets, mini-capsules, sachets, powder, granules, beverage mix, or liquid suspension. In some embodiments, the composition is loaded into a capsule, which may be a hard gelatin capsule or a soft gelatin capsule. In some embodiments, the composition is a powder of a plurality of particles (multiple particulates), wherein each of the particles comprises the core and the first polymer coating. In some embodiments, the composition is a plurality of mini-tablets or beads or mini-capsules. In some embodiments, the coated inorganic salt is in the form of a bead. In some embodiments, the dosage form is a sachet containing the coated inorganic salt in powder or granule form, suitable for reconstitution with water or a liquid medium prior to administration. In some embodiments, the dosage form is a beverage mix comprising the coated inorganic salt, a sweetener, and optionally a flavoring agent, configured to be mixed with water to form a drinkable electrolyte preparation. In some embodiments, the dosage form comprises the inorganic salt in an effective amount to provide a normal electrolyte level to a subject in need thereof. In some embodiments, the dosage form is free from other ingredients for therapeutic purposes. In some embodiments, the active agent that elicits any therapeutic or physiological effect in the dosage form consists essentially of the inorganic salt.

In some embodiments, the coated inorganic salt is formulated as a beverage mix powder that, when added to about 100 mL to about 1000 ml of water, produces a flavored electrolyte drink suitable for athletes, sportspersons, or individuals who are electrolyte deficient. In some embodiments, the beverage mix comprises the coated inorganic salt along with a sweetener such as acesulfame potassium, a flavoring agent such as orange, lemon, or fruit punch flavor, and optionally a gelling agent such as xanthan gum. In some embodiments, the beverage mix is packaged in a sachet or stick-pack for single-use administration. In some embodiments, the coated inorganic salt is formulated in a caplet form by compressing the coated granules or beads with suitable tableting excipients into a capsule-shaped tablet.

In some embodiments, the dosage form contains the inorganic salt in an effective amount to provide a normal electrolyte level to a subject who is electrolyte deficient. As used herein, an “effective amount” of the inorganic salt to provide a normal electrolyte level refers to an amount sufficient to restore or maintain the subject's electrolyte concentration within a range characteristic of healthy individuals. In some embodiments, the effective amount ranges from about 0.01 mg to about 100 g, from about 10 mg to about 100 g, from about 100 mg to about 50 g, from about 100 mg to about 10 g, or from about 500 mg to about 5 g of the inorganic salt per dosage form unit or per administration.

In some embodiments, the composition provides a certain amount of acid, ranging from about 0.001% to about 2%. In some embodiments, the amount of acid in the composition ranges from about 0.001% to about 0.01%, about 0.01% to about 0.1%, about 0.1% to about 1.0%, or from about 1% to about 2% in the composition. Nonlimiting examples of the amount include about 0.001%, about 0.005%, about 0.01%, about 0.05%, about 0.1%, about 0.5%, about 1%, about 1.5%, about 2%, and any range between any two of the aforementioned values.

In some embodiment, the acid can be citric acid, ascorbic acid tartaric acid, succinic acid, maleic acid, malic acid, or other poly-carboxylic acid. In some embodiments, the composition has a pH of between about 2 to about 8. In some embodiments, the composition has a pH of between ranges from about 2 to about 3.5, about 2.5 to about 4, about 3 to about 4.5, about 3.5 to about 5, about 4 to about 5.5, about 4.5 to about 6, about 5 to about 6.5, about 5.5 to about 7, about 6 to about 7.5, or about 6.5 to about 8. Nonlimiting examples of the amount include about 2, about 2.5, about 3, about 3.5, about 4, about 4.5, about 5, about 5.5, about 6, about 6.5, about 7, about 7.5, and about 8, and any range between any two of the aforementioned values.

In some embodiments, the composition comprises additionally an effervescence agent. In some embodiments, the effervescence agent is a carbon dioxide (CO₂) producing agent which, when present in the stomach or an acidic environment, will release CO₂. In some embodiments, the effervescence agent can be a combination of sodium bicarbonate and citric acid. In some embodiments, the effervescence agent can be a combination of sodium carbonate and citric acid.

Other excipients can also be included in the composition, combinations and liquid system disclosed herein, including for example plasticizer, flavor, taste enhancer, stabilizer. Nonlimiting examples of plasticizers include diethyl phthalate, tributyl citrate, triethyl citrate, glycerin, propylene glycol, polyethylene glycol, tributyrin. For instance, PEG 3350 at 10% of the enteric coating polymer level can be used. A solvent mixture containing ethanol, acetone and water at 50%, 20% and 30%, respectively can be used to facilitate the process of coating.

### Combinations

Another aspect of the disclosure provides the composition in the form of a combination comprising the coated inorganic salt(s) and a polyethylene glycol. Polyethylene glycol acts as a colon cleanser. The molecular weight of the polyethylene glycol may be for example, 100, 200, 400, 800, 1000, 1200, 1500, 1800, 2000, 2200, 2500, 2800, 3000, 3200, 3350, 3500, 3800, 4000, 4200, 4500, 4800, 5000, 5200, 5500, 5800, 6000, 6500, 7000, 8000, 10,000 daltons or any range between any two the aforementioned values. In one embodiment, the polyethylene glycol polymer has a molecular weight ranging from 2000 daltons to 8000 daltons. In a preferred embodiment, the polyethylene glycol polymer is PEG 3350 or PEG 8000.

In some embodiments, the combination may also comprise a flavoring agent. Non-limiting examples of flavoring agent include limited to peppermint oil, lemon, and fruit punch. In a further embodiment, the combination may also comprise a suspending agent selected from, but not limited to xanthan gum, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof.

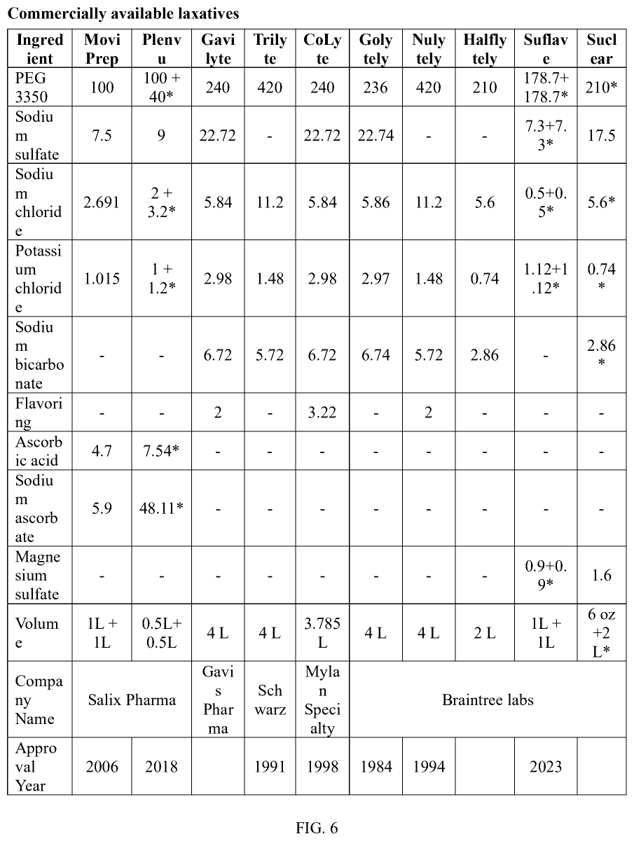

The combination may also comprise a commercially available laxative. In a further embodiment, the laxatives may be synthetic or derived from natural products, i.e., herbs and fibers. The laxatives may be used together with the combination or sequentially. Preferred examples of commercially available laxatives are selected from Gavilyte, Golytely, CoLyte, Nulytely, Trilyte, Halflytely, MoviPrep, Plenvu, Suflave, and Suclear. These laxatives and their respective ingredients are detailed in FIG. 6. In some embodiments, the coated inorganic salt(s) is admixed with a laxative, with non-coated salts present in the formulation. In some embodiments, the coated inorganic salt(s) is used in place of the non-coated salts present in the laxative.

### Liquid System

Another aspect of the disclosure provides the composition in the form of a liquid system comprising the coated inorganic salt(s) disclosed herein and a liquid medium admixed with the coated inorganic salt(s). The liquid system can be in a suspension, a viscous mixture or a gel, wherein the composition remains undissolved as a solid form.

In some embodiments, the liquid medium is pure water. In some embodiments, the liquid medium is polyethylene glycol. In some embodiments, the liquid medium is a combination of polyethylene glycol and water. In some embodiments, the molecular weight of the polyethylene glycol may be for example, 100, 200, 400, 800, 1000, 1200, 1500, 1800, 2000, 2200, 2500, 2800, 3000, 3200, 3350, 3500, 3800, 4000, 4200, 4500, 4800, 5000, 5200, 5500, 5800, 6000, 6500, 7000, 8000, 10,000 daltons or any range between any two the aforementioned values.

In some embodiments, the liquid medium comprises a suspending agent. Nonlimiting examples of suspending agents include xanthan gum, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and alike. In a preferred embodiment, the suspending agent is selected from xanthan gum or sodium carboxymethyl cellulose. The liquid system may further include water for adjusting the viscosity of the system.

The liquid system can be prepared by mixing the coated inorganic salt(s) with a suspending agent and diluent(s) in suitable proportions. Water may be added for viscosity adjustment or composition of a suspension or a gel. The kinematic viscosity was measured by the Canon viscometer. In some embodiments, the viscosity of the liquid system ranges from 10 cps to 2000 cps, from 50 cps to 1000 cps, 50 cps to 100 cps, 100 cps to 500 cps or 500 cps to 1000 cps. Nonlimiting examples of the viscosity include about 10, about 20, about 50, about 100, about 200, about 300, about 500, about 800, about 1000, about 1200, about 1500, about 2000 cps and an any range between any two of the aforementioned values. In some embodiments, the ratio by weight between the suspending agent and water ranges from 1:10 to 1:10000, from 1:50 to 1:5000, from 1:100 to 1:1000, from 1:200 to 1:500. In some embodiments, the ratio by weight between the suspending agent and water is 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 1:1000, 1:2000, 1:3000, 1:4000, 1:5000, 1:6000, 1:7000, 1:8000, 1:9000, 1:10000 or any range between any two of the aforementioned values. In a preferred embodiment, the ratio by weight between the suspending agent and water is between about 1:200 to about 1:500. In some embodiments, the suspending agent is present in an amount of about 0.01% to about 10% of the total composition. In a preferred embodiment, the suspending agent is present in an amount of about 0.2% to about 1.0% in the final gel formed.

In some embodiments, the liquid system is configured to reduce the volume of liquid to be administered to a subject. In some embodiments, the liquid system can be a gel or a semi-solid. In some embodiments, the volume of the liquid system ranges from about 0.1 liters to about 8.0 liters, about 0.1 liters to about 7.0 liters, about 0.1 to about 6.0 liters, about 0.1 to about 5.0 liters, about 0.1 to about 4.0 liters, about 0.1 to about 3.0 liters, about 0.1 to about 2.0 liters, about 0.1 to about 1.0 liters. In some embodiments, the liquid system has a total volume of about 1.0 liters, or any range between any two of the aforementioned values.

### 3. Kit

Another aspect of the disclosure provides a kit comprising (i) the coated inorganic salt(s) as detailed above and (ii) a liquid medium. In some embodiments, the liquid medium comprises a suspending agent and/or water. Nonlimiting examples of suspending agents include xanthan gum, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof.

In some embodiments, the liquid medium comprises a polyethylene glycol and/or water, wherein the polyethylene glycol is a colon cleansing agent. In some embodiments, the liquid medium is pure water. In some embodiments, the liquid medium is polyethylene glycol. In some embodiments, the liquid medium is a combination of polyethylene glycol and water. Polyethylene glycol acts as a colon cleansing agent. The molecular weight of the polyethylene glycol may be for example, 100, 200, 400, 800, 1000, 1200, 1500, 1800, 2000, 2200, 2500, 2800, 3000, 3200, 3350, 3500, 3800, 4000, 4200, 4500, 4800, 5000, 5200, 5500, 5800, 6000, 6500, 7000, 8000, 10,000 daltons or any range between any two the aforementioned values. In one embodiment, the polyethylene glycol polymer has a molecular weight ranging from 2000 daltons to 8000 daltons. In a preferred embodiment, the polyethylene glycol polymer is PEG 3350 or PEG 8000.

In some embodiments, the kit also comprises a flavoring agent, which may be added to any of the aforementioned components (i) or (ii), or as an additional component. Non-limiting examples of flavoring agent include limited to peppermint oil, lemon, and fruit punch.

In some embodiments, no gelling agent is added. The contents will be dissolved in water and coated electrolytes are swallowed along with the PEG solution.

In some embodiments, a gelling agent is added in (ii) along with other excipients. The contents when mixed with water form a nice gel. Coated electrolytes are swallowed along with the second gel.

In some embodiments, a gelling agent is added in (ii) along with other excipients. The contents when mixed with water form a nice gel. Coated electrolytes are suspended in the PEG gel. The mixture is swallowed within 15 to 20 minutes.

In some embodiments, Sodium sulfate and/or magnesium sulfate/citrate beads/granules are mixed with electrolytes.

In some embodiments, a marketed purgative product is administered along with PEG and electrolytes.

The kit may also comprise a commercially available laxative, which may be added to any of the aforementioned components (i) or (ii) or as an additional pouch. In a further embodiment, the laxatives may be synthetic or derived from natural products, i.e., herbs and fibers. Non-limiting examples of laxatives include bisacodyl, picosulfate sodium, senna extract, Isabgol, psyllium, docusate sodium, castor oil, and polycarbophil. The laxatives may be used together with the colonoscopy kit or sequentially. Preferred examples of commercially available laxatives containing polyethylene glycol are selected from Gavilyte, Golytely, CoLyte, Nulytely, Trilyte, Halflytely, MoviPrep, Plenvu, Suflave, and Suclear. These laxatives and their respective ingredients are detailed in FIG. 6. In some embodiments, the coated inorganic salt(s) is admixed with a laxative, with non-coated salts present in the formulation. In some embodiments, the coated inorganic salt(s) is used in place of the non-coated salts present in the laxative.

4. Method of Making Coated Inorganic salt(s)

Another aspect of the patent document provides a method of making the coated inorganic salt(s) disclosed herein. The method may include a step of preparing the salt core by mixing a salt or granule thereof with a diluent and a gelling agent. The salt core can include an inner core. Optionally, there is a step of coating the core with a coat of water-insoluble, but water-swellable polymer, such as ethyl cellulose. Subsequent steps include coating with an enteric coat or a reverse enteric coat and optionally applying an outer coat.

In some particular embodiments, the disclosure provides a method of making the coated inorganic salt(s) comprising sugar beads or suglets. In some embodiments, the method comprises: (a) an extrusion step; (b) a spherization step; and (c) loading electrolytes on sugar beads.

In some embodiments, the coated salts comprise a sugar bead or suglets. In some embodiments, the method comprises (a) loading sugar beads into a coating pan, (b) admixing inorganic salt(s) and a diluent or gelling agent with the sugar beads; (c) spraying a binder to adhere the inorganic salt(s) and diluent or gelling agent to the sugar beads. In some embodiments, the method further comprises drying the coated inorganic salt(s) at about 40° C. to 50° C. for 4 to 6 hours. In some embodiments, the method further comprises admixing the coated inorganic salt(s) with an undercoat, enteric coat, and/or reverse enteric coat. In some embodiments, the binder is Povidone K30.

In some embodiments, fluid bed dryer is used to coat the salts. In some embodiments, a top spray gun is used in the fluid bed process. In some embodiments, a bottom spray gun was used in the fluid bed process. In some embodiments, a pan coater is used in the process. In some embodiments, the coating is performed using a solution of polymer or a dispersion of polymer in water. In some embodiments, the process further includes color coating the coated inorganic salt(s). In some embodiments, sweeteners are included in the coating matrix.

In some embodiments, a tangential spray system in a conical rotor fluid bed processor is used to coat the salts and/or to prepare the sugar beads. The tangential spray conical rotor processor comprises:
(a) a conical rotor configured for controlled spiraling and helicoidal particle flow;
(b) a mechanically fixed slit air gap defined by machining and precise tolerances, as opposed to a floating or self-adjusting rotor mechanism;
(c) a tangential spray nozzle with a straight nozzle configuration for direct spray injection into the rotating particle bed;
(d) an external purging mechanism beneath the rotor to increase drying efficiency as the process gains weight and particle size; and
(e) PLC-controlled process parameters including inlet air volume, temperature, spray rate, rotor speed, and humidity. The conical rotor processor is capable of powder layering onto sugar bead cores, pellet formation from fine crystals, seal coating, and enteric coating.

Compared to conventional open coating pan techniques and conventional top-spray or bottom-spray fluid bed processes, the tangential spray conical rotor produces:
(a) improved process control and validation compared to open coating pan technique, which requires operator skill;
(b) reduced particle size variation, producing more uniform, spherical beads;
(c) improved yield of beads meeting specifications; and
(d) enhanced scalability for commercial manufacturing. The process parameters for tangential spray coating include: rotor speed selected to achieve controlled centrifugal motion and spiraling particle flow; inlet air temperature of about 30° C. to about 70° C.; spray rate controlled to prevent agglomeration while achieving uniform layering; atomization pressure optimized for the specific coating solution; and product temperature maintained at about 35° C. to about 55° C. during coating. In some embodiments, the tangential spray conical rotor is not essential, and equivalent beads may alternatively be produced using conventional top-spray or bottom-spray fluid bed equipment, though potentially with reduced uniformity and scalability.

In some embodiments, the method of preparing pure sugar beads comprises:
(a) providing sugar crystals having a particle size of about 0.5 mm to about 1.0 mm as seed cores;
(b) loading the sugar crystals into a fluid bed processor or coating equipment, preferably a tangential spray conical rotor processor;
(c) preparing a sugar syrup binder solution by dissolving sugar in water;
(d) applying sugar powder having a particle size of about 30 mesh to about 50 mesh onto the rotating sugar crystals while simultaneously spraying the sugar syrup binder solution to adhere the sugar powder to the crystals;
(e) continuing the layering process until the desired bead size is achieved; and
(f) drying the beads at about 40° C. to about 60° C. When water from the sugar syrup evaporates, it produces an additional sugar coating, thereby building up uniform, spherical, water-soluble beads entirely free of cornstarch or other water-insoluble components. The pure sugar beads are then loaded with one or more inorganic salts using a binding solution, followed by application of an undercoat, enteric coat, and optionally an outer coat as described herein.

In some embodiments, the method of loading electrolytes onto pure sugar beads using a tangential spray conical rotor processor comprises:
(a) loading the pure sugar beads into the conical rotor chamber;
(b) admixing inorganic salt(s) and optionally a diluent or gelling agent with the pure sugar beads;
(c) spraying a binder solution, such as Povidone K30 solution or sugar syrup, tangentially into the rotating particle bed to adhere the inorganic salt(s) to the sugar beads;
(d) controlling the rotor speed, inlet air temperature, spray rate, and atomization pressure via PLC programming to achieve uniform electrolyte loading with minimal agglomeration;
(e) drying the electrolyte-loaded beads at about 40° C. to about 50° C. for about 4 to about 6 hours; and
(f) applying one or more functional coats comprising an undercoat (e.g., HPMC E5), an enteric coat (e.g., HPMC Phthalate, Eudragit L100-55, or Eudragit L30D-55), and optionally an outer coat (e.g., PEG stearate or wax).

In a preferred embodiment for scale-up manufacturing, the method of making enteric-coated electrolyte pellets comprises the following four stages:
Stage 1—Preparation of Pure Sugar Pellets: Sugar is milled (pulverized) and sifted through a 150-mesh sieve. Sugar crystals of a desired particle size (e.g., 60-80 mesh for Golytely-type formulations or 16-25 mesh for Suprep-type formulations) are loaded into a tangential spray conical rotor processor (e.g., APCG 175). A sugar syrup binder solution (e.g., 50% w/w sugar in water) is prepared using an overhead mechanical stirrer. The sugar powder is loaded onto the starter sugar crystals using tangential spray of the sugar syrup binder in the conical rotor. The resulting pure sugar pellets are dried within the tangential rotor granulator with hot air until the moisture content is less than about 2%, preferably less than about 1%. The dried pellets are sifted to achieve a narrow particle size distribution and good pellet integrity. In some embodiments, the pure sugar pellets dissolve completely in water within approximately 1 minute, confirming that no insoluble residue remains.
Stage 2—Electrolyte Loading onto Sugar Pellets: The electrolyte mixture is pulverized and sifted through a 150-mesh sieve. The pulverized electrolyte powder is blended to achieve uniformity. The pure sugar pellets from Stage 1 are loaded into the tangential spray conical rotor processor (e.g., APCG 175). The electrolyte powder mixture is loaded onto the sugar pellets by tangential spray of a binder solution (e.g., PVP K30 solution or sugar syrup) in the conical rotor. In some embodiments, sugar syrup is used as the binder for the electrolyte loading step, providing the advantage that all ingredients remain water-soluble. The electrolyte-loaded pellets are dried within the tangential rotor granulator with hot air until the moisture content is less than about 2%. The dried pellets are sifted through appropriate sieves (e.g., 10, 12, 14, 16, 18, 20 mesh) to achieve the desired particle size distribution. Critical testing parameters at this stage include particle size distribution, moisture content, solubility, and assay of loaded electrolytes.
Stage 3—Application of Seal Coat: A seal coat solution is prepared comprising HPMC E5 and a plasticizer (e.g., PEG 6000 at about 10% of the HPMC weight) dissolved in purified water with heating. The seal coat is applied to the electrolyte-loaded pellets from Stage 2 using a bottom spray in the fluid bed processor (e.g., APCG 175 bottom spray configuration). The seal-coated pellets are dried until moisture content is less than about 2%. The seal coat provides a barrier between the electrolyte core and the enteric polymer, improving pellet robustness and preventing interaction between the electrolytes and the enteric coating polymer. Critical testing parameters at this stage include particle size distribution, moisture content, and solubility. In some embodiments, the seal-coated pellets produce a clear solution upon dissolution in water within approximately 3-4 minutes, confirming that all ingredients are water-soluble.
Stage 4—Application of Enteric Coat: An enteric coating solution is prepared using either (a) Eudragit L30D-55 (a 30% aqueous dispersion of methacrylic acid-ethyl acrylate copolymer) with triethyl citrate as plasticizer in purified water, or (b) HPMC Phthalate (hypromellose phthalate) with triethyl citrate as plasticizer in an organic solvent system (e.g., isopropyl alcohol and acetone). The enteric coat is applied to the seal-coated pellets from Stage 3 using a bottom spray in the fluid bed processor (e.g., APCG 175 bottom spray configuration). The enteric-coated pellets are dried until moisture content is less than about 2%. The enteric-coated pellets are sifted through appropriate sieves (e.g., 8, 10, 20 mesh) and packaged in aluminum pouches. Similar coating can alternatively be achieved by pan coating or using a fluid-bed dryer with top spraying. Critical testing parameters at this stage include particle size distribution, moisture content, solubility, assay of enteric-coated pellets, and dissolution performance in simulated gastric (pH 1.2) and intestinal (pH 7.2) conditions.

In some embodiments, the starter sugar crystal size is selected based on the target product. For Golytely-type formulations with sodium sulfate, sodium bicarbonate, sodium chloride, and potassium chloride, starter sugar crystals of about 60-80 mesh (about 180-250 μm) are used. For Suprep-type formulations with sodium sulfate, potassium sulfate, and magnesium sulfate, larger starter sugar crystals of about 16-25 mesh (about 710-1190 μm) are used to produce smoother spherical pellets with a final particle size in the 16-25 mesh range. Other particle sizes are acceptable and may be selected based on the desired final pellet diameter and swallowability requirements.

### 5. Method of Providing Coated Inorganic Salt(s) to a Subject

Another aspect of the patent document relates to a method of providing an inorganic salt(s) to a subject in need thereof. The method includes administering to the subject the coated inorganic salt(s), compositions, and/or kits disclosed herein. In some embodiments, the method reduces or eliminates the taste of the inorganic salt(s). In some embodiments, the subject has a lower-than-normal level of the inorganic salt(s). In some embodiments, the subject is electrolyte deficient. In some embodiments, electrolyte deficient refers to a deficiency in one or more inorganic salt(s).

In some embodiments, the inorganic salt(s), compositions, and/or kits is delivered to a subject prior to a colonoscopy procedure. For colonoscopy patients, the compositions and/or kits to be administered comprise polyethylene glycol (PEG). Further, water is a necessary component for colonoscopy preparations since the major cleansing effect is osmotic in nature. Thus, water may be consumed by the patient as part of the compositions and/or kits or in addition to the compositions and/or kits. In some embodiments, water is administered to the colonoscopy patient prior to administering the compositions and/or kits. In some embodiments, water is administered to the colonoscopy patient after administering the compositions and/or kits. In some embodiments, water is administered simultaneously with the compositions and/or kits to the patient.

The salt administered to the subject may range from about 0.01 mg to about 100 g, from about 10 mg to 100 g, about 100 mg to about 50 g, about 100 mg to about 10 g, about 500 mg to about 5 g, or as determined by a medical professional.

One of the purposes of coating inorganic salt(s) (electrolytes) is to mask the taste. For example, salt is necessary in our food products and it provides a good taste. But excess salt makes the food salty. In fact, one may vomit if he/she consumes too much salt at a time. Different salts or electrolytes taste different. For example, potassium chloride is less salty but imparts a bitter taste. Sodium bicarbonate has an unpleasant taste, and it has a slightly acidic in taste. The coated inorganic salt(s) and methods disclosed address this effectively.

Besides masking the taste of inorganic salt(s), the coated inorganic salt(s) disclosed herein can be configured such that delivery to certain segments of the gastrointestinal tract can be achieved for a variety of purposes and the profile and rate of release of salts for absorption can be controlled. These are commonly termed as “functional” coats. By way of example, if large quantities of sodium chloride are ingested in the commercially available tablet preparations, there will be an extremely high percentage of individuals that will have a noxious reaction to the ingestion. This is principally due to the high local concentrations of salt within the lumen of the stomach, which can activate a variety of neurohumoral mechanisms inducing very severe nausea and repetitive vomiting. The coated inorganic salt(s) and methods disclosed herein speak to the administration of very high quantities of sodium chloride and many other inorganic salt(s), which can be released after the stomach and into the more capacious small intestine allowing for a variety of therapeutic advantages.

In some embodiments, the enteric coated salts provide a delayed release profile in the GI tract. In some embodiments, the enteric coated salts provide a delayed and sustained release profile in the GI tract.

In some embodiments, the coated inorganic salt(s) is configured to provide an immediate release, delayed release, sustained release or any combination thereof. In some embodiments, the coated inorganic salt(s) is configured to provide an immediate release of the inorganic salt(s). In some embodiments, the coated inorganic salt(s) is configured to provide an extended or sustained release of the inorganic salt(s). In some embodiments, the coated inorganic salt(s) is configured to provide a delayed release followed by an extended or sustained release of the inorganic salt(s). In some embodiments, the coated inorganic salt(s) is configured to provide a delayed release followed by an immediate release of the inorganic salt(s).

### Enumerated Embodiments

The following enumerated embodiments are provided, the numbering of which is not to be construed as designating levels of importance.

Embodiment 1. A composition of one or more inorganic salt(s), comprising (a) a core comprising the one or more inorganic salt(s); (b) a first polymer coating layer enclosing the core; wherein the inorganic salt(s) range from about 5% to about 95% by weight in the composition.

Embodiment 2. The composition of Embodiment 1, wherein the one or more inorganic salt(s) are admixed with a diluent or a gelling agent in the core; (c) the blend of an inorganic salt(s), diluent and a gelling agent is converted to form granules or beads.

Embodiment 3. The composition of Embodiment 1, wherein the inorganic salt(s) is in the form of a plurality of granules in the core.

Embodiment 4. The composition of Embodiment 3, wherein the inorganic salt(s) granules have an average particle size smaller than 2000 microns.

Embodiment 5. The composition of Embodiment 2, wherein the diluent or the gelling agent comprises one or more of polyethylene glycol, gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), corn starch, and carboxy methylcellulose, preferably the gelling agent is present in the core and swells upon contact with water thereby releasing the inorganic salt(s).

Embodiment 6. The composition of Embodiment 2, wherein the gelling agent is present in the core and swells upon contact with water thereby slowly releasing the inorganic salt(s).

Embodiment 7. The composition of Embodiment 1, wherein the core further comprises an inner core free from the inorganic salt(s), wherein the inorganic salt(s) are coated on the inner core.

Embodiment 8. The composition of Embodiment 1, further comprising an extended-release layer between the core and the first polymer coating layer, wherein the composition is configured to provide an extended release of the inorganic salt(s) over at least 1 hour.

Embodiment 9. The composition of Embodiment 8, wherein the extended-release layer comprises one or more of ethyl cellulose, sodium polyacrylate, polyacrylamide copolymer, ethylene maleic anhydride copolymer, crosslinked carboxymethyl cellulose, polyvinyl alcohol copolymer, cross-linked polyethylene oxide, gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), corn starch, and carboxy methylcellulose.

Embodiment 10. The composition of Embodiment 1, wherein the first polymer coating is an enteric coating.

Embodiment 11. The composition of Embodiment 1, wherein the first polymer coating is a reverse enteric coating.

Embodiment 12. The composition of Embodiment 1, further comprising an undercoat between the core and the first polymer coating layer.

Embodiment 13. The composition of Embodiment 1, wherein the composition is free from water-insoluble polymers.

Embodiment 14. The composition of Embodiment 1, further comprising a second polymer coating enclosing the core and the first polymer coating.

Embodiment 15. The composition of Embodiment 1, wherein the second polymer comprises wax, PEG stearate coating, and any combination thereof.

Embodiment 16. The composition of Embodiment 1, which is in the form of a tablet.

Embodiment 17. The composition of Embodiment 1, which is in the form of a plurality of particles, wherein each of the particles comprises the core and the first polymer coating.

Embodiment 18. The composition of Embodiment 1, wherein the one or more inorganic salt(s) are selected from the group consisting of sodium salt, potassium salt, calcium salt, iron salt, copper salt, zinc salt, manganese salt, magnesium salt, molybdenum salt, cobalt salt, and chromium salt.

Embodiment 19. The composition of Embodiment 1, wherein the one or more inorganic salt(s) comprise one or more anions selected from the group consisting of chloride, fluoride, sulfate, bisulfate, carbonate, bicarbonate, and phosphate.

Embodiment 20. The composition of Embodiment 1, wherein the first polymer coating is configured to mask the taste of the inorganic salt(s).

Embodiment 21. The composition of Embodiment 1, which is configured to provide a delayed and/or extended release of the inorganic salt(s).

Embodiment 22. The composition of Embodiment 1, which is color coated.

Embodiment 23. A liquid system comprising the composition of Embodiment 1 and a suspending agent admixed with the composition.

Embodiment 24. The liquid system of Embodiment 23, wherein the suspending agent is selected from xanthan gum, polyethylene glycol, sodium alginate, carboxymethyl cellulose, methyl cellulose, hydroxypropyl methylcellulose, hydroxypropyl cellulose.

Embodiment 25. The liquid system of Embodiment 23, which is a gel.

Embodiment 26. A method of providing an inorganic salt(s) to a subject in need thereof, comprising administering to the subject the composition of Embodiment 1.

Embodiment 27. The method of Embodiment 26, which reduces or eliminates the taste of the inorganic salt(s).

Embodiment 28. The method of Embodiment 26, wherein the subject has a lower-than-normal level of the inorganic salt(s).

Embodiment 29. A method of preparing a subject for colonoscopy, comprising administering to the subject the liquid system of Embodiment 23.

Embodiment 30. A colonoscopy composition comprising:
(a) one or more electrolytes;
(b) a core comprising the one or more electrolytes; and
(c) a first polymer coating layer enclosing the core; wherein the electrolytes range from about 5% to about 95% by weight in the composition, preferably the electrolytes range from about 5% to about 50%.

Embodiment 31. The colonoscopy composition of Embodiment 30, wherein the colonoscopy composition is in the form of a plurality of granules, wherein each of the granules comprises the core and the first polymer coating.

Embodiment 32. The colonoscopy composition of any one of Embodiments 30 to 31, wherein the granules have an average particle size smaller than 3000 microns.

Embodiment 33. The colonoscopy composition of any one of Embodiments 30 to 32, wherein the first polymer coating is either an enteric coating or a reverse enteric coating.

Embodiment 34. The colonoscopy composition of any one of Embodiments 30 to 33, further comprising an undercoat between the core and the first polymer coating layer.

Embodiment 35. The colonoscopy composition of any one of Embodiments 30 to 34, wherein the colonoscopy composition is free from insoluble polymers.

Embodiment 36. The colonoscopy composition of any one of Embodiments 30 to 35, further comprising a second polymer coating enclosing the core and the first polymer coating, wherein the second polymer comprises wax, PEG stearate coating, and any combination thereof.

Embodiment 37. The colonoscopy composition of any one of Embodiments 30 and 32 to 36, which is in the form of a powder.

Embodiment 38. The colonoscopy composition of any one of Embodiments 30 to 37, wherein the one or more electrolytes are inorganic salt(s) selected from the group consisting of sodium salt, potassium salt, calcium salt, iron salt, copper salt, zinc salt, manganese salt, magnesium salt, molybdenum salt, cobalt salt, and chromium salt, and/or the inorganic salt(s) comprise one or more anions selected from the group consisting of chloride, fluoride, sulfate, bisulfate, carbonate, bicarbonate, phosphate, citrate.

Embodiment 39. The colonoscopy composition of any one of Embodiments 30 to 38, wherein the one or more electrolytes are admixed with a diluent or a gelling agent in the core, wherein the diluent or the gelling agent comprises one or more of polyethylene glycol, gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), and carboxy methylcellulose.

Embodiment 40. The colonoscopy composition of any one of Embodiments 30 to 39, wherein the diluent or the gelling agent is present in the core and swells upon contact with water thereby releasing the inorganic salt(s).

Embodiment 41. The colonoscopy composition of any one of Embodiments 30 to 40, further comprising an extended-release layer between the core and the first polymer coating layer, wherein the colonoscopy composition is configured so that, when tested with a USP type 2 dissolution system (Paddle Apparatus) at 50 rpm and a temperature of 37±0.5° C. in 125 mL 0.1 N HCl, at a pH of about 1.2, less than 1%, less than 5%, less than 10%, less than 20%, less than 30%, or less than 40% of the salt is released within 30 minutes, within 60 minutes, within 90 minutes, or within 120 minutes.

Embodiment 42. The colonoscopy composition of any one of Embodiments 30 to 40, further comprising an extended-release layer between the core and the first polymer coating layer, wherein the colonoscopy composition is configured so that, tested with a USP type 2 dissolution system (Paddle Apparatus) at 50 rpm and a temperature of 37±0.5° C. in 125 mL of 0.05 M phosphate buffer, at a pH of about 6.8 or a pH of about 7.4,
(b) from about 0% to about 10%, about 0% to about 20%, about 0% to about 30%, about 0% to about 40%, (e.g. 0%, 5%, 10%, 15%, 20%, 25%, 30%, 40% or any range between any two of the aforementioned % values) of the salt is released within 30 minutes;
(c) from about 30% to about 40%, about 30% to about 50%, about 30% to about 60%, about 30% to about 70% (e.g. 30%, 35%, 40%, 45%, 50%, 55%, 60%, 65%, 70% or any range between any two of the aforementioned % values) of the salt is released within 60 minutes; and
(d) from about 60% to about 70%, about 60% to about 80%, about 60% to about 90%, about 60% to about 100% (e.g. 60%, 65%, 70%, 75%, 80%, 85%, 90%, 95%, 100% or any range between any two of the aforementioned % values) of the salt is released within 90 minutes.

Embodiment 43. The colonoscopy composition of Embodiments 41 or 42, wherein the extended-release layer comprises one or more of ethyl cellulose, sodium polyacrylate, polyacrylamide copolymer, ethylene maleic anhydride copolymer, crosslinked carboxymethyl cellulose, polyvinyl alcohol copolymer, and cross-linked polyethylene oxide.

Embodiment 44. The colonoscopy composition of any one of Embodiments 30 to 42, which is configured to reduce or eliminate the taste of the electrolytes.

Embodiment 45. The colonoscopy composition of any one of Embodiments 39 to 44, wherein:
(a) the one or more electrolytes comprise one or more salts selected from the group consisting of sodium chloride, potassium chloride, sodium bicarbonate, sodium sulfate and magnesium sulfate;
(b) the first polymer coating layer enclosing the core is a methacrylic acid copolymer; wherein the electrolytes range from about 5% to about 25% by weight in the composition; and
(c) the diluent is sorbitol
(d) the gelling agent is carboxymethyl cellulose.

Embodiment 46. The colonoscopy composition of any one of Embodiments 30 to 45, further comprising a polyethylene glycol, and optionally a suspending agent admixed with the composition.

Embodiment 47. The colonoscopy composition of Embodiment 46, wherein the composition is in the form of a liquid, wherein the suspending agent selected from, but not limited to xanthan gum, polyethylene glycol, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof, wherein the polymer-coated core is suspended in the liquid.

Embodiment 48. The colonoscopy composition of Embodiment 47, which is in the form of a gel.

Embodiment 49. A kit comprising two pouches, wherein:
(a) a first pouch comprising the colonoscopy composition of any one of Embodiments 30 to 45, and
(b) a second pouch comprising a polyethylene glycol.

Embodiment 50. The kit of Embodiment 49, wherein the polyethylene glycol polymer has a molecular weight ranging from 2000 daltons to 8000 daltons.

Embodiment 51. The kit of Embodiment 49 or 50, further comprising a flavoring agent.

Embodiment 52. The kit of any one of Embodiment 49 to 51, further comprising a suspending agent selected from, but not limited to xanthan gum, polyethylene glycol, sodium alginate, sodium carboxymethyl cellulose, carboxymethyl cellulose, methyl cellulose, hydroxypropyl methylcellulose, hydroxypropyl cellulose, and any combination thereof.

Embodiment 53. The kit of any one of Embodiments 49 to 52, wherein the kit further comprises a laxative, or mixtures thereof.

Embodiment 54. The kit of Embodiment 53, wherein the laxative contains additional salts that are not coated.

Embodiment 55. The colonoscopy composition of any one of Embodiments 46 to 48, wherein the polyethylene glycol polymer has a molecular weight ranging from 2000 daltons to 8000 daltons.

Embodiment 56. The colonoscopy composition of any one of Embodiments 46 to 48 and 55, wherein the combination comprises a flavoring agent(s).

Embodiment 57. The colonoscopy composition of any one of Embodiments 45 to 47, 55 and 56, wherein the combination further comprises a laxative(s).

Embodiment 58. The colonoscopy composition of Embodiment 57, wherein the laxative contains additional salts that are not coated.

Embodiment 59. A method of providing electrolytes to a subject in need thereof, comprising administering to the subject the colonoscopy composition of any one of Embodiments 30 to 48 and 55 to 58, or administering to the subject the kit of any one of Embodiments 49 to 54.

Embodiment 60. The method of Embodiment 59, wherein the subject has an electrolyte imbalance.

Embodiment 61. The method of Embodiment 59 or 60, wherein the subject has a lower-than-normal level of electrolytes.

Embodiment 62. A balanced electrolyte composition comprising:
(a) one or more electrolytes;
(b) a core comprising the one or electrolytes; and
(c) a first polymer coating layer enclosing the core; wherein the electrolytes range from about 20% to about 95% by weight in the composition.

Embodiment 63. The electrolyte composition of Embodiment 62, wherein the one or more electrolytes are admixed with a diluent or a gelling agent in the core.

Embodiment 64. The electrolyte composition of Embodiment 63, wherein the electrolytes are in the form of a plurality of granules in the core.

Embodiment 65. The electrolyte composition of Embodiment 64, wherein the granules have an average particle size smaller than 2000 microns.

Embodiment 66. The electrolyte composition of Embodiment 63, wherein the diluent or the gelling agent comprises one or more of polyethylene glycol, gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), corn starch, and carboxy methylcellulose.

Embodiment 67. The electrolyte composition of Embodiment 63, wherein the gelling agent is present in the core and swells upon contact with water thereby slowly releasing the electrolytes.

Embodiment 68. The electrolyte composition of Embodiment 62, wherein the core further comprises an inner core free from the inorganic salt(s), wherein the electrolytes are coated on the inner core.

Embodiment 69. The electrolyte composition of Embodiment 62, further comprising an extended-release layer between the core and the first polymer coating layer, wherein the electrolyte composition is configured to provide an extended release of the electrolytes over at least 1 hour.

Embodiment 70. The electrolyte composition of Embodiment 69, wherein the extended-release layer comprises one or more of ethyl cellulose, sodium polyacrylate, polyacrylamide copolymer, ethylene maleic anhydride copolymer, crosslinked carboxymethyl cellulose, polyvinyl alcohol copolymer, and cross-linked polyethylene oxide.

Embodiment 71. The electrolyte composition of Embodiment 62, wherein the first polymer coating is an enteric coating.

Embodiment 72. The electrolyte composition of Embodiment 62, wherein the first polymer coating is a reverse enteric coating.

Embodiment 73. The electrolyte composition of Embodiment 62, further comprising an undercoat between the core and the first polymer coating layer.

Embodiment 74. The electrolyte composition of Embodiment 62, further comprising a second polymer coating enclosing the core and the first polymer coating.

Embodiment 75. The electrolyte composition of Embodiment 62, wherein the second polymer comprises wax, PEG stearate coating, and any combination thereof.

Embodiment 76. The electrolyte composition of Embodiment 62, which is in the form of a tablet or capsule.

Embodiment 77. The electrolyte composition of Embodiment 62, which is in the form of a powder.

Embodiment 78. The electrolyte composition of Embodiment 77, wherein the powder is dissolved in a liquid.

Embodiment 79. The electrolyte composition of Embodiment 62, which is in the form of a plurality of particles, wherein each of the particles comprises the core and the first polymer coating.

Embodiment 80. The electrolyte composition of Embodiment 62, wherein the one or more electrolytes are inorganic salt(s) selected from the group consisting of sodium salt, potassium salt, calcium salt, iron salt, copper salt, zinc salt, manganese salt, magnesium salt, molybdenum salt, cobalt salt, and chromium salt.

Embodiment 81. The electrolyte composition of Embodiment 62, wherein the one or more electrolytes are inorganic salt(s) comprise one or more anions selected from the group consisting of chloride, fluoride, sulfate, bisulfate, carbonate, bicarbonate, phosphate.

Embodiment 82. The electrolyte composition of Embodiment 62, wherein the first polymer coating is configured to mask the taste of the electrolytes.

Embodiment 83. The electrolyte composition of Embodiment 62, further comprising a suspending agent or gelling agent, wherein the suspending agent or gelling agent is gellan gum, xanthan gum, hydroxypropyl methylcellulose, hydroxypropyl methylcellulose, hydroxypropyl cellulose, lactose, mannitol, sorbitol, methyl cellulose, carboxymethyl cellulose, sodium carboxymethyl cellulose, Kollidon (povidone), corn starch, and carboxy methylcellulose.

Embodiment 84. The electrolyte composition of Embodiment 62, further comprising an effervescent agent.

Embodiment 85. A liquid system comprising the electrolyte composition of Embodiment 62 and a suspending agent admixed with the composition.

Embodiment 86. The liquid system of Embodiment 85, wherein the suspending agent is selected from, but not limited to xanthan gum, polyethylene glycol, sodium alginate, sodium carboxymethyl cellulose, carboxymethyl cellulose, methyl cellulose, hydroxypropyl methylcellulose, hydroxypropyl cellulose, acacia, agar, bentonite, carbomers, polyvinylpyrrolidone K30, and any combination thereof.

Embodiment 87. The liquid system of Embodiment 85, which is a gel.

Embodiment 88. The liquid system of Embodiment 85, wherein total volume is about 0.5 liter to about 1 liter.

Embodiment 89. The method of Embodiment 85, wherein the electrolyte composition is effective in suppressing the taste of the electrolytes.

Embodiment 90. A method of providing electrolytes to a subject, the method comprising administering to the subject the balanced electrolyte composition of Embodiment 62.

Embodiment 91. The method of Embodiment 90, wherein the balanced electrolyte composition is in the form of a gelling tablet.

Embodiment 92. The method of Embodiment 90, wherein the balanced electrolyte composition is in the form of effervescent electrolyte tablets.

Embodiment 93. The method of Embodiment 90, wherein the balanced electrolyte composition is a sprinkle formulation.

Embodiment 94. The method of Embodiment 90, wherein the balanced electrolyte composition is effective in suppressing the taste of the electrolytes.

Embodiment 95. The method of Embodiment 90, wherein the subject has an electrolyte imbalance.

Embodiment 96. The method of Embodiment 90, wherein the subject patient suffers from dysphagia.

Embodiment 97. The method of Embodiment 90, wherein the balanced electrolyte composition is a delayed-release formulation.

Embodiment 98. A method of preparing a subject for a colonoscopy procedure, comprising:
(a) providing a composition comprising a plurality of coated inorganic salt beads, wherein each bead comprises:
   (i) a pure sugar bead inner core that is substantially free of cornstarch, wherein the pure sugar bead inner core consists essentially of one or more sugars selected from the group consisting of maltose, sucrose, glucose, fructose, lactose, and combinations thereof,
   (ii) one or more inorganic salts coated on the inner core,
   (iii) optionally an undercoat, and
   (iv) an enteric coating layer enclosing the inorganic salts;
(b) orally administering the coated inorganic salt beads to the subject by either
   (i) placing the beads in the mouth in dry form and ingesting the beads with a liquid comprising water and/or a polyethylene glycol solution, or
   (ii) dispersing or suspending the beads in a liquid medium comprising polyethylene glycol, a suspending agent, and/or water to form an ingestible preparation, and administering the ingestible preparation to the subject; and
(c) optionally administering additional liquid to the subject, wherein the enteric coating prevents release of the inorganic salts in the stomach and the inorganic salts are released in the small intestine to maintain electrolyte balance during colonoscopy preparation.

Embodiment 99. The method of Embodiment 98, wherein the composition is in the form of beads, tablets, minitablets, capsules, granules, or powder.

Embodiment 100. The method of Embodiment 98, wherein the pure sugar bead inner core is prepared by coating sugar powder onto sugar crystals using sugar syrup as a binder.

Embodiment 101. The method of Embodiment 98, wherein the enteric coating comprises hydroxypropyl methylcellulose phthalate (HPMC Phthalate), a methacrylic acid copolymer, or a combination thereof.

Embodiment 102. The method of Embodiment 98, wherein all components of the coated inorganic salt beads are water-soluble such that no insoluble residue remains in the colon following administration.

Embodiment 103. The method of Embodiment 98, wherein the one or more inorganic salts are selected from the group consisting of: (i) sodium chloride, potassium chloride, sodium sulfate, and sodium bicarbonate; or (ii) sodium sulfate, potassium sulfate, and magnesium sulfate.

Embodiment 104. The method of Embodiment 98, wherein the coated inorganic salt beads are manufactured using a tangential spray system in a conical rotor fluid bed processor.

Embodiment 105. A method of preparing a subject for a colonoscopy procedure, comprising:
(a) reconstituting a dry composition with water to form a gel or liquid preparation, wherein the dry composition comprises:
   (i) coated inorganic salt beads having a pure sugar bead core that is free of cornstarch,
   (ii) polyethylene glycol, and
   (iii) a suspending agent;
(b) orally administering the gel or liquid preparation to the subject;
(c) administering additional clear liquid to the subject; and
(d) repeating steps (b) and (c) until the prescribed volume is consumed, wherein the coated inorganic salt beads release the inorganic salts in the small intestine and the composition is free of water-insoluble residue that could obscure colonic mucosa.

Embodiment 106. A coated inorganic salt composition for colonoscopy preparation, comprising:
(a) a pure sugar bead inner core consisting essentially of sucrose and being substantially free of cornstarch;
(b) one or more inorganic salts coated on the inner core;
(c) optionally, an undercoat; and
(d) an enteric coating layer comprising hydroxypropyl methylcellulose phthalate (HPMC Phthalate), wherein all components are water-soluble.

Embodiment 107. A method of manufacturing coated inorganic salt beads for colonoscopy use, comprising:
(a) preparing pure sugar beads by coating sugar powder onto sugar crystals using sugar syrup as a binder in a fluid bed processor;
(b) loading one or more inorganic salts onto the pure sugar beads using a binding solution;
(c) applying an undercoat;
(d) applying an enteric coating comprising hydroxypropyl methylcellulose phthalate (HPMC Phthalate) and/or a methacrylic acid copolymer; and
(e) drying the coated beads, wherein one or more of steps (a) through (d) is performed using a tangential spray in a conical rotor processor.

Embodiment 108. The method of Embodiment 98, wherein the coated inorganic salt beads are administered in dry form followed by administration of a liquid comprising water and/or a polyethylene glycol solution.

### EXAMPLES

### Example 1

Triethyl citrate was added as a plasticizer. PEG 3350 or Glyceryl monostearate make the coating softer. In batch 3, only PEG 3350 at 10% of the enteric coating polymer level was used. A solvent mixture containing ethanol, acetone and water at 50%, 20% and 30%, respectively was used in batch 4. Sodium chloride granules were coated with 10% Eudragit L100-55 and used blue color to identify the electrolyte. Sodium chloride was passed through 20 mesh before using in the batch. The volume of coating solution was thicker than desired. In batch 5, Eudragit concentration in the coating solution was reduced from 10% to 5%. The spraying process improved significantly.

Baking soda was in a fine powder form, which was very hard to coat. A solution of 2.03 g HPMC E5 in 15.31 g water was prepared. It was used to granulate 100 grams of baking soda (batch 7). The dough was passed through a 14-mesh sieve. The granules were dried in an oven at 40° C. and then transferred to Fluid Bed dryer for full drying and coating. 5% Eudragit was used to coat and the concentration of Eudragit in the coating solution was 4%.

In another batch, baking soda was granulated with Eudragit L100-55. The granules turned out to be satisfactory.

The baking soda with xanthan gum were granulated. Xanthan gum (0.5 g) was wetted with 0.5112 g of 200 proof ethanol. Water, 30 g, was added to xanthan gum and sonicated. About 50 g of baking was granulated with a sufficient quantity of xanthan gum solution. The mass was passed through a 14-mesh sieve and dried in the oven at 60° C.

In another batch, sodium bicarbonate was first granulated with Eudragit L100-55 dissolved in ethanol and acetone (70:30). The granules were passed through a 14-mesh sieve and dried in oven at 40° C. The granules with coated with Eudragit L100-55. Bigger granules were less colored suggesting lower coating. It is important to have a uniform particle size. Smaller particles were observed to be get coated better, but there was higher % loss of smaller particles to the filter of fluid bed dryer. Table 1 lists the result of sieve analysis of coated sodium bicarbonate.

TABLE 1
Analysis of coated particles

| | Particle size in | Weight of particle | |
|---|---|---|---|
| Mesh size | microns | over the sieve | Percent |
| 20 | 841 | 7.07 | 7.69 |
| 30 | 595 | 12.22 | 13.29 |
| 40 | 420 | 15.60 | 16.97 |
| 50 | 297 | 15.44 | 16.80 |
| Below 50 mesh | Less than 297 | 40.26 | 43.79 |

In general, it is desired to get all particles below 420 microns as they are coated better.

### Example 2

For batch 8, first baking soda particles were granulated with xanthan gum. The granules were dried at 50° C. overnight. Particles were passed through 14 mesh. Some of the particles were crushed with a mortar and a pestle. Also conducted small trial where different size particles of NaHCO3 were added to 0.1 N HCl. They showed similar amount of effervescence. It seemed that more xanthan gum was needed for granulation. All the granules were passed through 30 mesh. Half of these could pass through 40 mesh. The total weight of granules was 94.55 g. The coating solution contained 4% Eudragit L100-55. The % coating was 10% of the weight of granules. Two color shades were observed with larger particles having lighter shade.

### Example 3

Potassium chloride (KCl) was obtained in a granular form. In batch 10, KCl granules were coated as is. The color yellow was used to differentiate from other coated granules. A 10% coating was used and the granules showed nice uniform yellow color.

In batch 12, 74.99 g of sodium sulfate was weighed and added 25 grams of mannitol. Powders were mixed well in a zip-lock bag. The mixture was granulated with xanthan gum gel in water. Wet mass was passed through size 14 mesh. The granules and the coating were satisfactory.

### Example 4

In Batch 14, 5% Eudragit concentration in the coating solution was used. Following is the composition of coating solution (Table 2).

TABLE 2
Coating Solution for FB Batch #14 with
NaCl (100 gm), 15% Polymer

| Ingredients | Amount | |
|---|---|---|
| Eudragit L100-55 | 15.0 | gm |
| PEG 3350 | 1.5016 | gm |
| Ethanol | 141.83 | gm |
| Acetone | 56.85 | gm |
| Water | 85.08 | gm |
| Blue color | 28.3 | mg |

It produced a nice coating and there was a very slight salty taste to the granules.

### Example 5

### Batch 15: Coating of Sodium Bicarbonate.

The granulation solution was prepared using 1040 mg xanthan gum in 25 mL water. Sodium bicarbonate (151.2 g) was mixed with 48.63 g mannitol. Powders were mixed well and passed through 20 mesh sieves. The powder mixture was granulated with the xanthan gum gel. The wet mass was passed through a 14-mesh sieve and dried at 50° C. The coated granules had satisfactory properties.

### Example 6

### Batch 16: Coating of Potassium Chloride.

Weighed 109.75 μm of KCl, passed through a 20-mesh sieve, then passed through a 80-mesh sieve. Granules on the top of 80 mesh sieve (100.27 μm) were used for coating. The coating process was excellent and no lumps were observed in the granules. The granules were slightly salty, but could feel a slight bitter taste of potassium chloride.

### Example 7

### Sieve Analysis of New Sodium Sulfate (Table 3)

TABLE 3
Results of sieve analysis of sodium sulfate powder

| | Particle | | Weight | % passed |
|---|---|---|---|---|
| Mesh | size in | Weight on | passing | through the |
| size | microns | top of sieve | through sieve | sieve |
| 14 | 1410 | 0 | 49.42 | 98.78 |
| 20 | 841 | 0.09 | 49.37 | 98.68 |
| 30 | 595 | 1.70 | 47.58 | 95.10 |
| 40 | 400 | 8.82 | 38.54 | 77.03 |
| 60 | 250 | 9.42 | 29.05 | 58.07 |

A stack of sieves was prepared in the order listed in Table 3 above. Sodium sulfate powder, 50.03 g, was placed on 14 mesh. The stack was vibrated to let the powder pass through sieves gently. Nearly all the powder passed through 14 and 20 mesh. It means, all the particles were smaller than 841 microns. Only about 5% stayed on 30 mesh, which suggested that most particles were smaller than 595 microns. About 42% powder passed through 60 mesh (250 microns) and 58% remained on the top. In general, most of the sodium sulfate particles were less than 500 microns.

Clearly, sodium sulfate had to be granulated to increase its particle size. First, gels of different polymers were used to granulate sodium sulfate. Four different compositions were selected to granulate. Batch G1—weighed 25.2 g of sodium sulfate in a wide-mouth beaker. On a hot plate @ setting 2.5, heated and kept adding HPMC solution (2.5 g in 36 mL) with stirring. Batch G2, 25 g sodium sulfate+2.501 g HPC-mixed in a Ziplock bag. Transferred to a beaker. Added HPC gel with stirring. Batch G3-25 g sodium sulfate+2.5 g HPC in a Zip-lock bag. Transferred to a beaker and granulated with diluted HPC solution. Batch G4-25 g sodium sulfate+0.68 g HPC EXF Pharm+4.47 g lactose. It was granulated with a gel prepared by dissolving Blanose 7M31 in water. All the granules were passed through 14 mesh sieves. The granules were dried at 50° C. overnight and again passed through the 14-mesh sieve. In about 20 mL water, added a pinch of granules from each batch. Batch G1—granules stayed together. Batches G2 and G3—All the particles came together and formed a lump. Batch G4—All the granules stayed separated and then dissolved. Based on the data, it was decided to granulate 100 g sodium sulfate with 3 g of HPC and 22 g of lactose. Two coats were applied to sodium sulfate granules. The first coat was with a polymer solution containing 9.97 g of Blanose 7LF g in 251.5 mL water. In FB batch 17, the granules were first coated with Blanose. The batch was finished and the percent yield was 71.36%. The Blanose coating was followed by the enteric coating. Green color was used in the enteric coating solution.

### Example 8

### Batch 18-Sustained Release Sodium Sulfate Granules.

Bottom spray in the fluid bed dryer was used. Blanose 7 LF, 10 grams, was dissolved in 80:20 water: ethanol mixture. Blanose solution was coated on 122.8 grams of sodium sulfate granules. The percent yield was 86.95%. Table 4 lists the composition of Eudragit L100-55 solution used in coating sodium sulfate granules.

TABLE 4
Composition of Enteric coating solution
for FB Batch # 18- Na2SO4

| Ingredients | Actual amount | |
|---|---|---|
| Eudragit L100-55 | 15.1 | gm |
| Triethyl Citrate | 1.51 | gm |
| Ethanol | 141.24 | gm |
| Acetone | 57.75 | gm |
| Water | 85.31 | gm |
| Green Color | 25.4 | mg |

The % yield for the batch was 87.05%.

### Example 9

### Granulation of Sodium Bicarbonate—

To 100 grams of Sodium bicarbonate was added 3 g of HPC and 22 g lactose. Mixed and granulated. Granules were dried at 50° C. and were passed through a 14-mesh sieve. The granules had discolored slightly. Thus, sodium bicarbonate may show some instability in the presence of lactose. When added to 0.1 N HCl, it showed effervescence. Undercoat solution-10 g Blanose CMC 7 LF in 280 mL water plus 70 mL ethanol. The enteric coating solution contained triethyl citrate as the plasticizer. The yield was 88.95%

As sodium bicarbonate showed interaction with lactose, it was decided to use HPC as the diluent in batch 20. 100 g sodium bicarbonate and 11.04 g of Klucel EXF were granulated. Granules were passed through a 14-mesh sieve. Granules were dried at 50° C. overnight. Dried granules were pure white. Passed the granules through 14 mesh again. 4% Eudragit L100-55 solution was used to enteric coat granules (Table 5). The percent coating was about 15%. The yield of coated granules was 85.84%.

TABLE 5
Enteric Coating of FB Batch #20, NaHCO3 granules with HPC

| Ingredients | Actual amount (gm) |
|---|---|
| Eudragit L100-55 | 14.28 |
| Ethanol | 158.59 |
| Acetone | 63.58 |
| Water | 97.34 |
| Aspartame K | 0.1001 |
| Honey Flavor | 0.54 |
| Triethyl Citrate | 1.49 |

### Example 10

Batch 21: Coating of Sodium Chloride with Ethyl Cellulose

Ethyl cellulose is not soluble in water, but dissolves in methanol, ethanol etc. organic solvents. For 10% coating on 100 g sodium chloride, 10 grams of ethyl cellulose was needed. Ten grams of ethyl cellulose did not dissolve easily in 200 mL of 200 proof ethanol. The effect of % water in ethanol on the solubility of ethyl cellulose was studied. Prepared the coating solution using-10 g ethyl cellulose, 1-gram triethyl citrate, FD&C Blue color 30 mg in 20% water in ethanol solution. The coating process was smooth and the % yield was 87.3%.

### Example 11

Batch 22—Coating of KCl with Ethyl Cellulose

KCl granules were smaller than NaCl granules. KCl was granulated first. KCl, 100 grams was mixed with 11 g HPC Klucel EXF in a ziplock bag. Granulating solution contained-Sodium CMC 7LF—2 grams and PEG 400—202.2 mg in Water. KCl with HPC mixture was granulated with the granulating solution. The mass was passed through 12-mesh sieve and then dried at 40° C. overnight. Table 6 lists the composition of the coating solution.

TABLE 6
Coating solution of FB Batch #22, KCl

| Ingredients | Actual amount (gm) |
|---|---|
| Ethocel | 10.01 |
| Ethanol | 200 |
| Water (16.7%) | 40 |
| Triethyl Citrate (10%) | 1.0 |
| Yellow color | 30.2 mg |

The granulation process was smooth and the yield was 90.6%.

### Example 12

Batch 23—Coating of Sodium Sulfate with Ethyl Cellulose

Sodium sulfate powder was mixed with HPC and the mixture was granulated (Table 7).

TABLE 7
Composition for the Granulation of Na2SO4

| Ingredients | Actual amount | |
|---|---|---|
| Na2SO4 | 100.30 | mg |
| HPC | 11.11 | gm |
| Granulating solution | 10.32 | gm |

Passed through 12 mesh sieve and kept it at 40 °C for drying.

Table 8 lists the ingredients used in the composition of the coating solution

TABLE 8
Coating solution composition for sodium sulfate

| Ingredient | Amount | |
|---|---|---|
| Ethanol 200 proof | 200 | mL |
| Water | 40 | mL |
| Ethyl cellulose | 10.01 | g |
| Triethyl citrate | 1.03 | g |
| FD&C Green 3 | 29.8 | mg |

The coating process went smooth and the % yield was 90.8%

Coating of Sodium Bicarbonate Granules with Ethyl Cellulose

No color was used in the coating solution to make the coated granules white. The process went well and the yield was 89.8%.

### Example 13

Batch 25: Coating of Sodium Chloride with Ethyl Cellulose with HPMC Undercoat.

TABLE 9
Composition used in the granulation of NaCl

| Ingredient | Amount, gm | Actual, gm |
|---|---|---|
| NaCl (85%) | 170 | 170.02 |
| Klucel EXF HPC (15%) | 15 | 15.00 |
| Sorbitol | 15 | 15.08 |

Sodium chloride, Klucel and sorbitol were first passed through 20 mesh and then mixed well in a zip lock bag (Table 9).

### Example 14

TABLE 10
Granulating solution with Sodium CMC
used in sodium chloride granules

| Ingredient | Amount | |
|---|---|---|
| Aqualon CMC 7LF | 1.0190 | gm |
| PEG 400 | 0.170 | gm |
| Water | 25.21 | gm |

Water heated to get a slightly thick gel. About 12.19 μm of this solution was mixed with the powder and kneaded. Passed through 14 mesh sieve and kept at 40° C. for drying.

HPMC coating solution-9.53 g of HPMC was dissolved in 151.53 mL water. The coating process was completed in 2 hours and the coating was satisfactory.

### Example 15

Ethyl Cellulose Coating of Sodium Chloride with HPMC Undercoat

TABLE 11
Composition of ethyl cellulose coating solution. 4 gm

| Ingredient | Percent | Actual | |
|---|---|---|---|
| Ethanol | 75.8 | 301.68 | gm |
| Water | 18.95 | 77.68 | gm |
| Ethyl Cellulose | 4.74 | 19.01 | gm |
| Triethyl Citrate | 0.47 | 1.91 | gm |
| Blue Color | 0.014 | 55.4 | mg |

Weight of coated NaCl: 208.14 μm; % Yield: 100%.

Placed a pinch of coated salt in about 20 mL water. Tasted after half an hour and an hour. No salty taste observed. Added 1 N HCl to the sample and no salt particles dissolved after one hour. After overnight at RT, the water from the sample was salty.

In another sample, a pinch of coated granules was placed in about 20 mL Sorensen buffer, pH was 7.40. The granules did not dissolve in half hour and the solution was not salty. The solution was salty next day.

### Example 16

### Batch 26: Coating of Sodium Bicarbonate Granules

Granulation with HPC and Sorbitol

TABLE 12
Composition used in the Granulation of NaHCO3

| Ingredient | % | Actual, gm |
|---|---|---|
| NaHCO3 | 85 | 100.04 |
| Klucel EXF HPC (15%) | 7.5 | 8.83 |
| Sorbitol | 7.5 | 8.83 |
| Total | 100 | 117.7 |

Passed all materials (Table 12) through 20 mesh. Mixed well in a zip lock bag.

TABLE 13
Composition for the Sodium CMC solution

| Ingredients | Actual | |
|---|---|---|
| Aqualon CMC 7LF | 1.0190 | gm |
| PEG 400 | 0.170 | gm |
| Water | 25.21 | gm |

PEG 400 was added to hot water. CMC was added slowly to get a slightly thick gel. About 9.36 μm of this solution was mixed with the powder and kneaded. Passed through 20 mesh and kept at 60° C. for drying.

### PEG Stearate Coating:

Prepared a PEG Stearate solution in water-ethanol mixture (Table 14). It was used to coat enteric-coated granules of sodium bicarbonate.

TABLE 14
Composition of PEG Stearate coating solution

| Ingredient | Actual | |
|---|---|---|
| PEG Stearate | 5.75 | gm |
| Water | 49.09 | gm |
| Ethanol | 50.48 | gm |

### Preparation of Beads of Electrolytes

The following methods describe the preparation of spheres of electrolytes. Two methods for making spheres were used—1. Extrusion and Spheronization and 2. Loading electrolytes on sugar beads.

### Example 17

### Extrusion Spheronization

This involved four steps—1. Mixing and Granulation of an electrolytes with suitable excipients, 2. Extrusion—It was performed with an extruder, 3. Spheronization—It was performed with a spheronizer and 4. Drying—the beads formed were dried at 40° C. to 50° C.

The granulation step was similar to the granulation performed earlier. The key was to bind the materials but should not be too sticky. The core contained a diluent, an electrolyte and a gelling agent. The bead size after spheronization was between 1 to 4 mm. These beads were coated with HPMC E5 as an undercoat. The beads were then enteric coated and then coated with PEG Stearate.

### Example 18

Preparation of Sodium Bicarbonate Beads with Sugar Pellets

Sodium bicarbonate powder, hydroxypropyl cellulose, Povidone K30, and HPMC E5 were passed through a 40-mesh sieve. Povidone K30 was dissolved in water to make a binding solution. Sugar beads were loaded in the coating pan. Sodium bicarbonate and HPC were mixed and were added to the sugar beads in small portions. Povidone K30 solution was sprayed carefully so that the mixture of sodium bicarbonate and HPC gets coated on sugar beads. The pellets were dried at about 40° C. to 50° C. for 4 to 6 hours. These coated beads were coated further with HPMC E5. HPMC E 5 works as an undercoat for the enteric coat or a reverse enteric coat. The beads were also coated with ethyl cellulose or other water-insoluble/water-swellable polymers

### Example 19

### Dissolution Studies on Salts

USP Apparatus II (paddle) is used to conduct dissolution studies of coated electrolytes. Three media are used—distilled water, 0.1 N HCl, and 0.05 M phosphate buffer, pH 6.8. The volume of each medium to be used is 125 to 900 mL. The paddle speed of 50 rpm is used. The sampling times for the dissolution studies in HCl are—10, 20, 30, 45 and 60 minutes. The sampling times for the dissolution studies in water and phosphate buffer are—15, 30, 60, 90, 120 and 180 minutes. One coated electrolyte is tested in each dissolution testing. Sodium is a common ion in sodium chloride, sodium sulfate and sodium bicarbonate. The other two cations are—potassium and Magnesium. ICP-MS technique is used to determine the levels of cations in the dissolution media.

When the electrolytes are enteric coated, a release of less than 40% electrolyte in the 0.1 N HCl medium was expected. In water as a dissolution medium or pH 6.8 phosphate buffer as the dissolution medium, the enteric coating will dissolve first. The gelling polymer in the core would swell and the electrolyte will dissolve over 2 to 3 hours. Q75% value would be about 1 hr (Q75 is the time to release 75% of the electrolyte).

The non-colonoscopy beads are coated with water-insoluble, water-swellable polymer coating. A release of less than 20% electrolyte in the 0.01 N HCl medium was expected. In water and in pH 6.8 buffer, the Q75% value will be about 2 hours.

### Example 20

### Taste Testing

The salts are taste tested by the volunteers. About 1 to 2 grams of coated electrolytes are administered to the subjects. They keep the salts in the mouth for 3 to 5 minutes and the taste is recorded. The salts are spit out and the mouth is rinsed with water. The taste of salt in the mouth is examined again after 3 to 5 minutes.

### Example 21

### Coating of a Mixture of Salts

A mixture of salts can be coated. Benefit of coating a mixture includes saving of time of coating, saving of cost, ease of handling materials during packaging etc. The mixture may include for example sodium chloride+potassium chloride, sodium chloride+sodium sulfate, sodium chloride+sodium bicarbonate, sodium chloride+potassium chloride+sodium sulfate, sodium chloride+potassium chloride+sodium sulfate+sodium bicarbonate, etc.

Sodium sulfate and sodium bicarbonate are commercially available in a fine powder form. Sodium sulfate, 170 grams, was mixed with 30 grams of sodium bicarbonate. It was mixed with 22 grams of HPC and granulated with the sodium CMC solution. The granules were dried in the oven at 40° C. The granules were coated with HPMC E5 (undercoat), Eudragit L100-55 (enteric coating) and PEG stearate.

### Examples 22-31

The following examples pertain to colonoscopy compositions. In all the following examples, the electrolytes mentioned were coated suitably to mask the taste and provided functional coats.

### Example 22

Compositions of Colonoscopy Batch #1 and #2 have been listed in Table 15.

TABLE 15
Compositions of Batches 1 and 2

| Ingredient | % |
|---|---|
| PEG 3350 | 86.03 |
| Sodium Sulfate | 8.29 |
| Sodium bicarbonate | 2.46 |
| Sodium chloride | 2.14 |
| Potassium chloride | 1.08 |
| Total | 100.0 |
| Water to dilute | 50 mL or 100 mL |

100 g batches of PEG 3350 and electrolytes was prepared as specified in Table 15. The contents were diluted to 500 mL and 1000 mL, respectively. Both batches were very salty, Batch 2 being less salty. An average dose of PEG 3350 is 260 grams. It means, the entire amount will be diluted to about 3 L and the final solution will be still salty. This explains the usage of about 4 L volume for the colonoscopy compositions. A sweetener (aspartame) and a flavoring agent (peppermint oil) were added to the solutions from batches 1 and 2 and it reduced the saltiness slightly. Xanthan gum was added to these batches, and it was determined that xanthan gum did not form a gel in the presence of high concentration of salts.

New batches were prepared using only PEG 3350 and different gelling agents-xanthan gum, kappa Carrageenan and gelatin. Gelatin needed heating at about 80° C. to dissolve fully. All formed satisfactory gels. In order to make a stable gel and also, to block salty taste, it was deemed necessary to coat the salts. Enteric (Eudragit L100/55, batch 8) and reverse entering (Eudragit EPO, batch 9) coatings were compared on sodium chloride granules. One gram of polymers dissolved in 10 mL of 200 proof ethanol. Yellow and blue colors were added for enteric and reverse entering coatings, respectively. Ten grams of sodium chloride granules were coated in a beaker under low heat and adding the coating solutions drop wise. The coated granules produced results as expected when the granules were added to 0.1 N HCl, water or pH 10 sodium hydroxide solution. The granules were tasted as is and showed slight salty taste. It means the coating were found to be effective.

### Example 23

Using the coated granules prepared, three batches of colonoscopy compositions were prepared (Table 16)

TABLE 16
Colonoscopy Batch #10, #11 and #12

| | Batch 10 | Actual | Batch 11 | Actual | Batch 12 | Actual |
|---|---|---|---|---|---|---|
| | Amount | amount | Amount | amount | Amount | amount |
| Ingredient | 29 g | (g) | 29 g | (g) | 29 g | (g) |
| PEG 3350 | 24 | 24.0088 | 24 | 24.0013 | 24 | 24.0480 |
| Sodium | — | — | — | — | 0.829 | — |
| Sulfate | | | | | | |
| Sodium | 4.29 | 4.2889 | — | — | — | — |
| Chloride | | | | | | |
| Enteric | — | — | 4.29 | 4.2896 | — | — |
| coated | | | | | | |
| Reverse | — | — | — | — | 4.29 | 4.2893 |
| enteric | | | | | | |
| coated | | | | | | |
| Xanthan | 0.60 | 0.6001 | 0.60 | 0.5995 | 0.60 | 0.6002 |
| gum | | | | | | |
| Peppermint | 0.04 | 0.0483 | 0.04 | 0.0586 | 0.04 | 0.0445 |
| oil | | | | | | |
| Acesulfame | 0.08 | 0.0802 | 0.08 | 0.0809 | 0.08 | 0.0803 |
| potassium | | | | | | |

All the powders were weighed in ziplock bags and mixed well. About 12 grams of each mixture was added to 30 mL water. Batch 10 was extremely salt, as expected and the pH of the solution was 6.21. Batch 11 was salty, but significantly less salty than batch 10. The sweetness was satisfactory, and the pH was 5.68. Batch 12 was less salty than batch 11 and the pH was 6.73.

In another set of dilutions, citric acid was added to water to bring the pH to about 3.5. Batch 10 was still salty. Batch 11, with enteric coating, was significantly less salty. Batch 12, with reverse enteric coating, became saltier as the pH was acidic and reverse enteric coating should dissolve in the acidic pH.

### Example 24

Plenvu® is one of the leading colonoscopy composition available in the market. It contains Dose 1 and Dose 2. Dose 1 contains one pouch, whereas Dose 2 has Pouches A and B. The total weight of pouch in Dose 1 is 115.96 grams, which contains—PEG 3350—100 g, Sodium sulfate—9 g, Sodium chloride—2 g, Potassium chloride—1 g. Dose 1 is dissolved in 16 oz (480 mL) of water. A 30-mL solution of Dose 1 of Plenvu was prepared. Various batches, which contained different amounts of aspartame was also prepared (Table 17).

TABLE 17
Effect of Aspartame on the taste of Plenvu

| | | Batch #14 | Batch #15 | Batch #16 | Batch #17 |
|---|---|---|---|---|---|
| | | (0.25% | (0.50% | (1% | (2% |
| Ingredients | Batch #13 | aspartame) | aspartame) | aspartame) | aspartame) |
| Aspartame | — | 12.6 mg | 25.3 mg | 50.8 mg | 100.3 mg |
| Plenvu | 5.0024 g | 5.026 g | 5.012 g | 5.027 g | 5.021 g |
| Solution | | | | | |
| Observations | Salty | Saltier | Tolerable | Too sweet | Did not |
| | but less | less sweet | sweet, not | | dissolve |
| | than | | salty | | fully |
| | NaCl | | | | |
| Sweetness | 0 | 3 | 5 | 10 | 10 |
| Saltiness | 8 | 4 | 1 | 0 | 0 |

Interestingly, a direct correlation between the amount of aspartame and saltiness was observed. As the amount of aspartame was increased, a decrease in the saltiness was observed. More than about 1% aspartame in the solution (not in the colonoscopy composition) cannot be added.

Plenvu Dose 2 pouch A contains PEG 3350—40 grams, sodium chloride—3.2 grams and potassium chloride—1.2 g and unknown excipients—1.86 g (total weight—46.26 g). Plenvu Dose 2 Pouch B contains sodium ascorbate—48.11 g and ascorbic acid—7.54 g (total weight—55.65 g). A 30 mL solution of Dose 2 was prepared by mixing appropriate amounts of powders from Pouch A and Pouch B. The solution had a slight saltiness, and it was moderately sweet.

A 30 mL salt solution with 750.4 mg sodium chloride, 2.25 g sodium sulfate and 75 mg of citric acid was prepared. 0.25% or less quantity of aspartame was added in the samples and their taste was checked. Similar to results for Plenvu Dose 1, the saltiness decreased as the amount of aspartame was increased. The sample was very salty without aspartame. In this study, a small percentage of aspartame was added so that the sweetness could be controlled (Table 18). More citric acid buffer was added to samples 18 and 19. In general, citric acid and aspartame addition to the formulation was observed to have a beneficial effect on taste of the formulation.

TABLE 18
Effect of Aspartame on salt solution.

| | Salt | | Actual | | | | |
|---|---|---|---|---|---|---|---|
| | solution | | amt | | | | |
| Batch | (g) | Aspartame | (mg) | Sweetness | Saltiness | Sourness | pH |
| # 18 | 5.015 | 0.25% | 12.7 | 5 | 5 | 1 | 3.00 |
| (0.25%) | | (12.5 mg) | | | | | |
| # 19 | 5.037 | 0.20% | 10.3 | 4 | 5 | 1 | 2.95 |
| (0.2%) | | (10 mg) | | | | | |
| # 20 | 5.008 | 0.15% | 7.7 | 3 | 6-7 | 1 | 3.00 |
| (0.15%) | | (7.5 mg) | | | | | |
| #21 | 5.037 | 0.10% | 5.7 | 2 | 8 | 1 | 2.96 |
| (0.1%) | | (5.0 mg) | | | | | |
| #22 | 5.025 | 0% | 0 | 0 | 10 | 1 | 2.87 |
| (0%0 | | | | | | | |

| | | | | | |
|---|---|---|---|---|---|
| #18* | 25mg Citric acid; | 5 | 4 | 3 | 3.15 |
| | 15.5 mg sodium citrate | | | | |
| #19* | 25mg Citric acid; | 4 | 5 | 1 | 3.36 |
| | 20 mg Sodium citrate | | | | |

### Example 25

The interaction of gelatin and PEG 3350 was examined. In control samples, 1 g PEG 3350 or 0.202 g of gelatin were dissolved in 5 mL water. PEG 3350 dissolved in water easily. Gelatin dissolved in water with ultrasonication and formed a Jello. In another two samples, 1 of PEG 3350 and 0.1 g or 0.2 g of gelatin were added to 5 mL water. With 0.1 g gelatin, PEG 3350 dissolved in water, but the gelatin partially dissolved. With 0.2 g gelatin, PEG 3350 dissolved but gelatin formed lumps. It seemed that PEG 3350 interacted with gelatin and may be crosslinking gelatin.

Various gelling agents, microcrystalline cellulose, methyl cellulose, Gellan gum, xanthan gum, guar gum, Kollidon 90F, Methocel and corn starch were examined for their gel formation and taste of the gel. All were observed to be good gelling agents and were tasteless. Some have sticky, gummy taste. Gel formation in the presence of PEG 3350 was examined (Table 19). Table 20 lists the observations from these batches.

### Colonoscopy Batches #28 to #32

TABLE 19
Compositions of batches 28 to 31

| Batch # | Amount | Batch #28 | Batch #29 | Batch #30 | Batch #31 |
|---|---|---|---|---|---|
| PEG 3350 | 1 g | 1.0402 g | 0.9971 g | 1.0004 g | 0.9946 g |
| X. Gum | 100 mg | 99.8 mg | — | — | — |
| Guar Gum | 50 mg | — | 52.5 mg | — | — |
| MC | 200 mg | — | — | 201.4 mg | — |
| A15LU | | | | | |
| Gellam | 100 mg | — | — | — | 102.4 mg |
| Gum low | | | | | |
| AC | | | | | |
| Water | 4 mL | 4.0353 + | 4.0297 g | 4.0118 g | 3.9930 g |
| | | 2.7482 | | | |

TABLE 20
Results of batches 28 to 31.

| Batch | Observations | Taste test |
|---|---|---|
| #28 | Formed a thick gel immediately. | Very thick and very sticky. |
| | No lumps. Lot of air bubbles. | Need less xanthan gum. |
| | | Need only 1.25%. Xanthan |
| | | gum taste acceptable. |
| #29 | Thin liquid. Many particles | Anhydrate particles. |
| | suspended in first 2 min. | Taste not good. |
| | Viscosity increased with time | |
| #30 | MC dispersed easily. PEG dissolved. | NA |
| | Turbid. Sufficient thickness. | |
| | Under microscope- undissolved, | |
| | unhydrated particles. | |
| #31 | Gellan gum dispersed easily. PEG | NA |
| | dissolved. Sample looked turbid. | |
| | Under microscope undissolved | |
| | particles. | |

Table 21 lists the composition of batch 32 and its observations. Sodium CMC showed a potential of being used in the colonoscopy formulations.

TABLE 21
Formulation Batch #32

| Ingredients | Amount | Batch #32 | Observations |
|---|---|---|---|
| PEG 3350 | 1 g | 0.9971 g | Na CMC dispersed easily. |
| Na CMC 10 | 150 mg | 150.5 mg | Gel slightly turbid. In 15 min, |
| | (3.75%) | | the gel became transparent. |
| Water | 4 mL | 3.9994 g | Taste was good. |

### Example 26

A typical colonoscopy composition was used and the effects of xanthan gum and sodium carboxymethyl cellulose as the gelling agents were examined (batches 35 to 37, Table 22).

TABLE 22
Effect of salt mixture on gel formation.

| | | | | Actual | | |
|---|---|---|---|---|---|---|
| | | | No | Batch | Actual | Actual |
| Ingredients | X.Gum | NaCMC | Salts | #35 (g) | #36 (g) | #37 (g) |
| PEG 3350 | 236 | 236 | 236 | 9.71 | 8.39 | 8.25 |
| Na2SO4 | 22.74 | 22.74 | — | — | 0.8155 | 0.7928 |
| NaHCO3 | 6.74 | 6.74 | — | — | 0.2435 | 0.2441 |
| NaCl | 5.86 | 5.86 | — | — | 0.2119 | 0.2012 |
| KCI | 2.97 | 2.97 | — | — | 0.1106 | 0.1016 |
| Xanthan Gum | 3.4 | — | 3.4 | 0.1445 | 0.1225 | — |
| Sodium CMC | — | 15 | — | — | — | 0.5238 |
| Ascorbic acid | 1 | 1 | 1 | 0.0402 | 0.0404 | 0.0308 |
| Citric acid | 1.65 | 1.65 | 1.65 | 0.0715 | 0.0603 | 0.0600 |
| Flavor | 1.4 | 1.4 | 1.4 | 0.0605 | 0.0506 | 0.0512 |
| Acesulfame | 0.96 | 1 | 0.96 | 0.0412 | 0.0347 | 0.0357 |
| Total | 281.76 | 293.36 | 243.45 | 10 g | 10 g | 10 g |

Weighed all the ingredients except salts and added to 35 mL water.

Batch #35 formed a thin gel and has a sweet and salty taste.

Batch #36 & 37 started breaking when salts were added to the solution. The pHs of batches were—#35:3.39, #36:6.36 and #37:6.43.

In another experiment, first the gel was formed using xanthan gum. Four electrolytes were added one at a time and checked the stability of the gel. Table 23 lists the composition of batch #38.

TABLE 23
Colonoscopy composition Batch #38.

| | Batch #38 | Actual Batch | |
|---|---|---|---|
| Ingredients | (g)/50 mL | #38 (g) | pH |
| PEG 3350 | 48.55 | 48.55 | |
| X. Gum | 0.70 | 0.7050 | |
| Ascorbic acid | 0.20 | 0.2001 | |
| Citric acid | 0.35 | 0.3524 | |
| Flavor | 0.30 | 0.3096 | |
| Acesulfame | 0.20 | 0.2027 | |
| Total | 50.3 | | |
| Coated KCI | 0.11 | 0.1120 | 3.19 |
| Coated NaCl | 0.21 | 0.2149 | 3.14 |
| Coated Na2SO4 | 0.81 | 0.8105 | 3.32 |
| Coated NaHCO3 | 0.24 | 0.2435 | 6.56 |

Weighed and added all the ingredients except salts. All the salts were enteric coated. After gel was formed, weighed and added coated KCl, NaCl, Na₂SO₄ and NaHCO₃ one by one and measured the pH before adding the next salt. Gel started breaking after adding Sodium sulfate. The pH of sample increased after adding sodium bicarbonate.

A batch of colonoscopy composition without xanthan gum was prepared (Table 24). PEG 3350, ascorbic acid, citric acid, acesulfame and fruit punch flavor were dissolved in water. The taste of product was satisfactory. First added enteric coated sodium chloride and enteric coated potassium chloride coated beads. The beads settled down. Added enteric coated sodium sulfate beads. All the salts settled down. In 2-3 minutes, salts agglomerated and formed a lump. The pH of solution was 3.4. It was found that a sodium sulfate coating dissolved and broke xanthan gum gel. This result was corroborated by making another batch without sodium sulfate and the gel did not break.

TABLE 24
Composition of colonoscopy composition without xanthan gum.

| Ingredients | Batch #39 (g) | Actual (g) |
|---|---|---|
| PEG 3350 | 8.39 | 8.3919 |
| Ascorbic acid | 0.04 | 0.0423 |
| Citric acid | 0.06 | 0.0628 |
| Acesulfame | 0.034 | 0.0343 |
| Flavor- Fruit punch | 0.05 | 0.0509 |
| Coated Na2SO4 (Batch #12) | 0.81 | 0.8109 |
| Coated NaHCO3 | 0.24 | - |
| Coated NaCl (Batch #14) | 0.21 | 0.2108 |
| Coated KCI (Batch #16) | 0.11 | 0.1113 |

### Example 27

The interaction PEG 3350 with various gelling agents was tested (Table 25).

Solubility and Viscosity values of PEG 3350 with Methocel E5 (HPMC); HPMC50 and Methyl Cellulose

TABLE 25
Effect of PEG 3350 of different gelling agents.

| | Theoretical | With Methocel | With | With |
|---|---|---|---|---|
| Ingredients | amounts | E5 | HPMC50 | Methylcellulose |
| PEG 3350 | 29.5 g | 29.5061 g | 29.5022 g | 29.5042 g |
| Polymer | 625 mg | 625.3 mg | 6265.5 mg | 625.3 mg |
| Citric Acid | 150 mg | 150.3 mg | 150.5 mg | 150.0 mg |
| H20 | 125 mL | 125 mg | 125 mg | 125 mg |

In all the samples, the polymers used did not dissolve and formed lumps. The role of citric acid to acidify the formulation by bringing the pH to about 3.5.

Xanthan gum and gellan gum at 5 mg/mL were added to water and added citric acid at 1.2 mg/mL. Both did not form gel.

Various gelling agents (MC, CMC, Sodium CMC, Gellan gum, Xanthan gum, HPMC E5, HPMC E50) were used to attempt to form a gel in the presence of citric acid. 62.5 mL water, 325 mg gelling agent and 62.5 mg of citric acid. pH was maintained at 4.0.

The gel in water was first prepared, then citric acid was added into the gel (Table 26). Less citric acid was added in some samples. Sodium CMC formed a thick gel, thus additional water was added for the.

TABLE 26
Effect of citric acid on various gelling agents.

| | MC | CMC | NaCMC | Gellam | HPMC | HPMC | X.gum |
|---|---|---|---|---|---|---|---|
| Ingredient | (mg) | (mg) | (mg) | gum (mg) | E5 (mg) | E50 (mg) | (mg) |
| Water | 62.0 | 62.0 | 123.8 | 62.0 | 62.0 | 62.0 | 62.0 |
| Gelling agent | 327.4 | 327.2 | 324.9 | 324.9 | 326.5 | 324.2 | 325.0 |
| Citric acid | 62.5 | 103.4 | 95.4 | 50.0 | 51.8 | 52.2 | 66.3 |

After standing overnight at room temperature, following are the observations for various samples—

MC-clear, but watery, CMC—clear, but watery, Sodium CMC—clear, slightly viscous, Gellan gum-viscous, flowing, but cloudy, HPMC E5-clear and watery, HPMC E50—clear and watery (slightly viscous), Xanthan gum—Most viscous compared to others, flowing, slightly, turbid. More citric acid was added to bring down the pH of each gel below 4. The addition of enteric coated sodium sulfate granules (1 g each) destabilized the gels. Granules suspended only in the samples with xanthan gum and gellan gum. In gellan gum, granules clumped together to form a lump. After 5 hrs, the sample with xanthan gum was tasted. It produced a distinct taste of sodium sulfate showing partial dissolution of granules.

### Example 28

The formula of the colonoscopy preparation—Gravilyte G was used as the base composition to prepare batch 41 (Table 27). Gravilyte G contains 236 g of PEG 3350, 22.74 g of sodium sulfate, 6.74 g of sodium bicarbonate, 5.86 g of sodium chloride and 2.97 g of potassium chloride. All ingredients in a fine powder form were used in Powder 1. In Powder 2 mixture, coated granules were used. In Powder 1, xanthan gum is mixed in PEG 3350. Ascorbic acid and citric acid were mixed to the above mixture. This was followed by acesulfame and fruit punch flavor. The mixture was mixed well in a zip-lock bag. In a 100 mL bottle, added 87.5 mL water and mixed Powder 1 mixture. It formed a nice gel upon shaking. The taste of this product was very good. Mixtures for all four coated electrolytes were prepared (Table 28). The batch was stable for 30 minutes. As time passed, sodium bicarbonate produced effervescence and the pH increased to 6.42. All granules became soft and floated on the surface. The percentage of colon cleansing agents and coated electrolytes can be adjusted as needed.

TABLE 27
Composition of colonoscopy composition, batch 41

| Ingredient | Percent | For 25 g | Actual, g |
|---|---|---|---|
| Powder 1 | | | |
| PEG 3350 | 78.82 | 19.71 | 19.7218 |
| Xanthan gum clear | 1.64 | 0.41 | 0.4091 |
| Ascorbic acid | 0.38 | 0.09 | 0.0945 |
| Citric acid | 0.56 | 0.14 | 0.143 |
| Acesulfame | 0.32 | 0.08 | 0.0862 |
| Fruit punch | 0.47 | 0.12 | 0.1239 |
| Powder 2 | | | |
| Na2SO4 | 11.41 | 2.85 | 2.8503 |
| NaHCO3 | 3.38 | 0.85 | 0.8812 |
| NaCl | 1.97 | 0.49 | 0.4993 |
| KCI | 1.03 | 0.26 | 0.2694 |
| Total | 100 | 25 | |

Table 28 lists the compositions of Batches #42 to #45. We wanted to study the effect of various polymers on the gel formation.

TABLE 28
Composition of colonoscopy compositions, batch 42 to 45.

| | Batch 42 | | Batch 43 | | Batch 44 | | Batch 45 | |
|---|---|---|---|---|---|---|---|---|
| | | Actual, | | Actual, | | Actual, | | Actual, |
| Ingredient | Theo. | g | Theo. | g | Theo. | g | Theo. | g |
| PEG 3350 | 19.71 | 19.77 | 19.71 | 19.74 | 19.71 | 19.76 | 19.71 | 19.76 |
| No | — | — | — | — | — | — | — | — |
| Polymer | | | | | | | | |
| MC | — | — | 0.65 | 0.6585 | — | — | — | — |
| CMC | — | — | — | — | 0.65 | 0.6543 | — | — |
| HPMC E50 | — | — | — | — | — | — | 0.65 | 0.6505 |
| Ascorbic | 0.09 | 0.0950 | 0.09 | 0.0921 | 0.09 | 0.0915 | 0.09 | 0.0923 |
| Acid | | | | | | | | |
| Citric acid | 0.14 | 0.1409 | 0.14 | 0.1402 | 0.14 | 0.1418 | 0.14 | 0.1440 |
| Acesulfame | 0.08 | 0.798 | 0.08 | 0.0796 | 0.08 | 0.0783 | 0.08 | 0.0802 |
| Flavor | 0.12 | 0.1209 | 0.012 | 0.1214 | 0.12 | 0.1201 | 0.12 | 0.1214 |
| Water | | 87.69 | | 87.74 | | 87.76 | | 87.33 |
| Na2SO4 | 2.85 | 2.8508 | 2.85 | — | 2.85 | — | 2.85 | — |
| granules | | | | | | | | |
| NaHCO3 | 0.85 | 0.8588 | 0.85 | 0.8502 | 0.85 | 0.8479 | 0.85 | 0.8566 |
| granules | | | | | | | | |
| NaCl | 0.49 | 0.4921 | 0.19 | 0.4900 | 0.49 | 0.4954 | 0.49 | 0.4986 |
| granules | | | | | | | | |
| KCI | 0.26 | 0.2571 | 0.26 | 0.2598 | 0.26 | 0.2613 | 0.26 | 0.2620 |
| granules | | | | | | | | |
| Total | 24.59 | | 25.89 | | 25.89 | | 25.89 | |

Observations: Batch #42: Added to water. PEG dissolved in a minute with gentle shaking. The liquid became hazy and watery and the pH was 3.13. Acesulfame and fruit punch were added and the pH was 5.69. Addition of citric acid decreased the pH to 2.63. The system was still clear. The salts were added one by one. KCl powder settled, no yellow color to solution. Then added NaCl—no lumps, pH—3.03. NaHCO3—no effervescence. Sodium bicarbonate seemed to start reacting in few minutes and the pH increased to 4.07. Sodium sulfate granules were added to a bottle and shaken. The pH increased to 5.91 with a lot of effervescence. Sodium bicarbonate reacted with acids to produce effervescence. As acid was consumed, the pH of the system increased.

Batch #43: Transferred the powder mixture to a zip lock bag. Mixed well and added to water, shook for a minute. Uniformly distributed. No lumps, hazier than batch #42. Good sweetness and flavor, not very salty. pH 3.19. Slightly more viscous than batch #42.

Batch #44: same as batch #43. Hazy and slightly viscous than batch #42.

Batch #45: Same viscosity as batch #43 and #44. Hazy, this batch was more clear than other three formulations.

All these formulations had similar viscosity values and were equally hazy.

Instead of citric acid, 0.1 M and 0.25 M citrate buffers were used to prevent fluctuations in the pH with sodium bicarbonate. The pH is stable with 0.25 M citrate buffer, but the solution becomes too sour, and this concentration cannot be used.

### Example 29

Three batches 46, 47 and 48 were prepared (Tables 29 and Table 30). Batch #46 contained xanthan gum. Batch #47 did not contain xanthan gum. These batches contained vanilla and lemon flavors respectively.

TABLE 29
Colonoscopy Compositions, Batches #46 and #47

| | Batch 46 | | Batch 47 | |
|---|---|---|---|---|
| | Amount | Actual, | Amount | Actual, |
| Ingredient | (600 mL) | g | (600 mL) | g |
| PEG 3350 | 141.60 | 141.61 | 141.60 | 141.62 |
| X.gum | 2.95 | 2.9505 | - | - |
| clear | | | | |
| Ascorbic | 0.68 | 0.6821 | 0.68 | 0.6824 |
| acid | | | | |
| Citric acid | 0.68 | 0.6785 | 0.68 | 0.6838 |
| Acesulfame | 0.57 | 0.603 | 0.17 | 0.176 |
| FD&C | 0.08 | 0.086 | 0.008 | 0.0081 |
| Yellow | | | | |
| Vanilla | 0.84 | - | - | - |
| Lemon | - | - | 0.84 | 0.8408 |
| Flavor | | | | |
| Total | 147.41 | | 144.05 | |

#46—clear transparent, thickened gel. Little sourness, not sweet enough, no gummy or sticky feeling.

#47—dissolved easily but formed a turbid solution. Not sweet, lemon flavor is not so good. It was too strong. Some acidic touch.

Batch #48 did not contain citric acid, a coloring agent and a flavoring agent.

TABLE 30
Colonoscopy composition batch #48.

| | Batch 48 | | | |
|---|---|---|---|---|
| | | Amount | Amount | Actual, |
| Ingredient | Amount | (1 L) | (300m L) | g |
| PEG 3350 | 8.39 | 236 | 70.80 | 70.81 |
| X.gum clear | 0.175 | 4.92 | 1.48 | 1.4812 |
| Ascorbic acid | 0.04 | 1.13 | 0.34 | 0.3433 |
| Acesulfame | 0.068 | 1.91 | 0.57 | 0.5738 |
| Total | 8.673 | 243.96 | 73.19 | |

The batch prepared a nice thick gel, which was too sweet.

Blanose CMC 7HCF was used as the gelling agent (Table 31). It is a neutral molecule.

TABLE 31
Colonoscopy composition, Batch #49

| Ingredient | Per 100 mL | Actual |
|---|---|---|
| Gel | | |
| PEG 3350 | 23.60 g | 23.70 g |
| Blanose CMC 7HCF | 0.4 g | 0.4070 g |
| Ascorbic acid | 115 mg | 116.7 mg |
| Acesulfame | 115 mg | 116.6 mg |
| Citric acid anhydrous | 115 mg | 114.8 mg |
| Enteric coated granules | | |
| Na2SO4 | 600 mg | 601.6 mg |
| NaHCO3 | 180.0 mg | 181.5 mg |
| NaCl | 100 mg | 99.8 mg |
| KCI | 50 mg | 50.2 mg |

All of the ingredients were mixed except enteric coated granules in a ziplock bag. Added 100 mL water in a 250 mL bottle. Added above powder and mixed well.

Taste was good-sweetness and sourness. pH of gel without granules 4.21.

All of the granules were mixed to the gel solution. Granules suspended nicely. pH was 5.18. Taste after 5 min: less salty and more bitter, pH was 6.08. Agglomeration of granules was observed. Viscosity of batch #49 was 159.6 cSt. It was good enough to suspend all granules.

pH of batch #49 with granules after 10 min was 6.36. That means, NaHCO3 granules reacted mostly with acid.

The granules were coated with PEG stearate and MCT (medium chain triglyceride). PEG stearate (604.8 mg) was mixed with MCT (1.073 g, 36.1% PEG Stearate in the solution) and heated to produce a solution. Enteric coated sodium chloride, potassium chloride, sodium bicarbonate and sodium sulfate granules were weighed and mixed. The mixture was coated with 3 drops of PEG Stearate-MCT solution. The effect of oil coating on the dissolution rate of these electrolytes was observed. Colonoscopy batch #50 did not have any citric acid (Table 32).

TABLE 32
Colonoscopy composition, Batch #50

| Ingredients | Per 50 mL | Actual |
|---|---|---|
| PEG 3350 | 11.8 g | 11.95 g |
| Blanose CMC 7H4CF | 0.2 g | 200.8 g |
| Acesulfame | 60 mg | 61.2 mg |
| Na2SO4 granule | 300 mg | 307.8 mg |
| NaHCO3 granules | 90 mg | 105.5 mg |
| NaCI granules | 50 mg | 60.2 mg |
| KCI granules | 25 mg | 28.6 mg |

Mixed MCT-PEG stearate coated granules with PEG 3350. Mixed Blanose and Acesulfame. Added 50 mL water. Mixed with spatula. No lumps formed, thick gel. Good taste. It was sweeter. Therefore, need to reduce acesulfame 40 mg. No salty taste. All granules settled down. The solution was turbid. After half hour, the granules dissolved partially.

An additional batch (batch #51, Table 33) was made without coating granules with MCT-PEG stearate. Make another batch #51 without addition of oil.

TABLE 33
Colonoscopy composition, Batch #51

| Ingredients | Actual |
|---|---|
| PEG 3350 | 11.90 g |
| Blanose CMC 7H4CF | 199.6 mg |
| Acesulfame | 38.6 mg |
| Na2SO4 granule | 304.1 mg |
| NaHCO3 granules | 113.8 mg |
| NaCl granules | 63.5 mg |
| KCI granules | 37.0 mg |

All of the ingredients were mixed except PEG 3350 first. Added PEG 3350, mixed. Added 50 mL water, mixed. PEG 3350 dissolved and solution, became much clearer than batch #51. Taste of batch #51 was more bitter than batch #50.

Batch #50 after overnight, not salty, bitter and turbid.

Batch #51 after overnight, slightly salty, but slightly bitter and clear.

PEG stearate has a bitter taste. Therefore, in order to determine the effect of % PEG stearate on the dissolution of electrolytes, a minimal amount was added. Prepared 3 batches—batch 52 has only MCT coating on the granules, batch 53 had a coating of a solution containing 20% PEG Stearate in MCT, and batch 54 had a coating of 50% PEG stearate in MCT. Table 34 lists compositions of these three batches.

TABLE 34
Colonoscopy Composition, Batches #52 to #54

| | Amount/50 mL | Batch 52 | Batch 53 | Batch 54 |
|---|---|---|---|---|
| Ingredient | Theoretical | MCT | 20% PEG stearate in MCT | 50% PEG Stearate in MCT |
| PEG 3350 | 11.9 g | 11.968 g | 11.979 g | 11.928 g |
| Blanosed | 200 mg | 201.2 mg | 202.0 mg | 201.3 mg |
| 7H4CF | | | | |
| Acesulfame | 25 mg | 24.8 mg | 24.4 mg | 25.6 mg |
| K | | | | |
| Na2SO4 | 300 mg | 299.mg | 300.2 mg | 304.5 mg |
| NaHCO3 | 100 mg | 109.7 mg | 102.6 mg | 103.4 mg |
| (Batch#17) | | | | |
| NaCI | 50 mg | 66.3 mg | 61.7 mg | 64.5 mg |
| (Batch #14) | | | | |
| KCI | 25 mg | 31.6 mg | 33.8 mg | 34.6 mg |
| (Batch #16) | | | | |
| MCT | 65 mg | 84.1 mg | - | - |
| (Batch #20) | | | | |
| MCT/PEG | 65 mg | - | 83.7 mg | 88.1 mg |
| Stearate | | | | |

Zero time: All three batches formed nice gels. Turbidity 54>53>52. All granules settled at the bottom. PEG Stearate imparted whiteness to formulation.

One hour: All salts except NaHCO3 dissolved in all formulations. Batch #54 is most turbid. All gels were good, not salty. Added a pinch of citric acid in #54. #54 was slightly bitter, may be due to excess PEG stearate. Therefore, 20% PEG stearate is preferable. Gel did not break with a small amount of citric acid, pH 4.22.

The effect of acidic pH on the stability of salts was examined (Table 35). Batch 55 contained citric acid and batch 56 was without citric acid. Zero time: The pH values of 55 and 56 batches were 5.21 and 7.30, respectively. There was no salty taste in both. Both batches had a sufficient sweetness and formed good gels. 5 min: no salty in both. Slight sourness in batch 55. The movement of few sodium bicarbonate granules due to effervescence was observed. 10 min: pH of #55-5.99, pH of #56-6.96. Both discolored to bluish green. Formulation #55 was salty, #56 was not salty, very slight bitterness.

30 min: #56 slightly salty. #55 saltier than #56 and more bitter.

1 hour: #56 more salty and slightly bitter. #55 more salty and bitter. Viscosity of both seemed to be lower than at zero time for both batches.

Overall, batch #56 seemed to be better than #55 as it showed less taste issues, if administered within 30 minutes.

TABLE 35
Colonoscopy Compositions, Batches #55 and #56

| | Amount/ | Actual, batch 55 | Actual, batch 56 |
|---|---|---|---|
| Ingredient | 50 mL | With citric acid | w/o citric acid |
| PEG 3350 | 12 g | 12.08 g | 12.08 g |
| Blanose 7H4CF | 200 mg | 208.1 mg | 207.9 mg |
| Acesulfame | 25mg | 28.4 mg | 25.1 mg |
| Citric acid | 60 mg | 71.6 mg | - |
| Na2SO4 granules | 1136 mg | 1140.6 mg | 1128.9 mg |
| NaHCO3 granules | 336 mg | 341.5 mg | 340.9 mg |
| NaCI granules | 292 mg | 296.7 mg | 301.6 mg |
| KCI granules | 148.5 mg | 145.5 mg | 144.9 mg |
| MCT/ 20% PEG | 320 mg | 320.9 mg | 328.1 mg |
| Stearate | | | |

If the pH is not acidic, xanthan gum can be used in the formulation instead of Blanose. Batch #57 with xanthan gum and without citric acid was prepared (Table 36).

TABLE 36
Colonoscopy Composition, Batch # 57

| | Amount/ | Actual, batch 57 |
|---|---|---|
| Ingredient | 50 mL | With xanthan gum |
| PEG 3350 | 12 g | 11.96 g |
| Xanthan gum clear 80 | 250 mg | 251.7mg |
| Acesulfame K | 25 mg | 25.6 mg |
| Na2SO4 granules | 1136 mg | 1137.0 mg |
| NaHCO3 granules | 336 mg | 339.0 mg |
| NaCl granules | 292 mg | 295.7 mg |
| KCI granules | 148.5 mg | 147.8 mg |
| MCT/25% PEG Stearate | 320 mg | 333.3 mg |

Similar to previous batches, all electrolytes were weighed and added to the MCT-PEG Stearate solution. The salts were mixed well to get coated with the solution. Weighed PEG 3350 and added to the salt mixture. Xanthan gum and acesulfame were added to the above mixture and mixed well. Added water and stirred to dissolve the mixture.

White suspension similar to #56. NaHCO3 granules were floating. No salty taste at zero time. The pH was 6.83.

5 min: no salty taste, but slightly bitter. NaHCO3 granules started to dissolve or settle at the bottom. All salts at the bottom

10 min: Getting more salty and more bitter than #56. Started seeing two layers.

25 min: pH 6.92. NaHCO3 granules still intact at the bottom, some Na2SO4 granules at the top. More watery, pretty bitter, not too salty. No NaCl and KCl granules. It means, NaCl and KCl granules must have dissolved in the solution.

The granules of electrolytes were coated with MCT-Cocoa butter mixture (15% and 85%, respectively). The salts were found to be fully wet with MCT-Cocoa butter and the coating was probably got transferred to PEG 3350. It means, this composition would not work.

TABLE 37
Colonoscopy Composition Batch #58

| | Amount/ | Actual, batch 58 |
|---|---|---|
| Ingredient | 50 mL | With Cocoa butter |
| PEG 3350 | 60 g | 59.95 g |
| Blanose 7H4CF | 1000 mg | 1.0114 g |
| Acesulfame K | 100 mg | 99.5 mg |
| Na2SO4 granules | 5.62 g | 5.80 g |
| NaHCO3 granules | 1.680 g | 1.690 g |
| NaCl granules | 1.460 g | 1.455 g |
| KCI granules | 0.745 g | 0.7468 g |
| 15% MCT/85% Cocoa butter | TBD | 1.0104 g |

Sodium bicarbonate was granulated with sorbitol and HPC. The granules were coated with HPMC, enteric coating and PEG stearate. Batches prepared with Blanose (batch 59) and Xanthan gum (batch 60) were compared (Table 38).

Colonoscopy Composition, Batches #59, #60

| | | Actual, batch 59 | Actual, batch 60 |
|---|---|---|---|
| Ingredient | Amount | With Blanose | With X.gum |
| PEG 3350 | 12 g | 12.01 g | 12.01 g |
| Blanose 7H4CF | 200 mg | 200.8 mg | - |
| Acesulfame K | 25 mg | 25.8 mg | 26.0 |
| X.gum clear 80 | 250 mg | - | 250.0 |
| NaHCO3 | 336 mg | 337.0 mg | 337.1 mg |
| (batch 26) | | | |
| NaCl | 148.5 mg | 154.6 mg | 148.6 |
| KCI | 292 mg | 291.9 mg | 295.3 |
| pH | | 7.8 | 7.53 |

In the beginning, only sodium bicarbonate was added to the batches. Both suspended granules of sodium bicarbonate very well. Potassium chloride and sodium chloride were then added.

TABL 39
Observations for batches 59 and 60.

| | Batch #59 | Batch #60 |
|---|---|---|
| Zero time | No salty, granules | No salty, granules suspended, |
| | suspended and | Turbid |
| | KCl/NaCl settled | |
| | at the bottom, clear | |
| 15 min | Granules on top, clear. | Granules suspended, turbid. |
| | Very slightly | More salty and bitter than |
| | bitter and salty. | #59. |
| 30 min | Clear. Very slightly | Saltier than #59. Granules |
| | bitter, salty. Blue | dissolved and agglomerated |
| | and white granules. | to form lumps. |
| 45 min | Slightly, salty and clear | More lumps, Saltier and |
| | | turbid. |
| 60 min | Tolerable saltiness, | Salty and bitter. More lumps. |
| | nearly all particles | Granules agglomerated. |
| | dissolved clear. | |

Based on the observations (Table 39), Batch #59 seemed to be more acceptable.

Table 40 lists the composition of the colonoscopy preparation, Batch 61 containing magnesium citrate and sodium picosulfate.

TABLE 40
Colonoscopy composition, Batch 61

| Ingredient | Amount |
|---|---|
| Coated magnesium citrate beads | 30.3 g |
| Picosulfate sodium, 10 mg tablet | One |

A tablet of sodium picosulfate, 10 mg, was prepared using a ready-to-use mixture of compressible lactose and magnesium stearate. Magnesium citrate beads were coated beads were prepared using methods described before. The ingredients were filled in a pouch along with sorbitol and a lemon flavoring agent. The patient consumes the mixture with water, soda, or any other suitable drink.

Table 41 lists the composition of the colonoscopy preparation, Batch 62.

TABLE 41
Colonoscopy composition, Batch 62

| Ingredient | Amount, g |
|---|---|
| PEG 3350 | 236 |
| Coated potassium chloride yellow granules | 4.46 |
| Coated sodium bicarbonate white granules | 10.11 |
| Coated sodium chloride blue granules | 8.79 |
| Coated sodium sulfate green granules | 34.11 |

Coated granules of four salts listed in Table 41 were prepared using the aforementioned process. The granules were color coded as described in the table. The mixture of coated granules was placed in a pouch. PEG 3350 along with sorbitol, gelling agent, an artificial sweetener and a flavoring agent were placed in another pouch. The patient adds the ingredients from the PEG 3350 pouch to 500 ml water and will stir well. It forms a thick gel. The mixture of coated granules was suspended in the gel and the suspension is administered to the patient. The empty bottle of gel is rinsed with more water and the rinsate is consumed.

In batch 63, instead of granules of salts, beads of various salts in the formulation were used.

Table 42 lists the colonoscopy formulation composition of batch 64.

TABLE 42
Colonoscopy composition, batch # 64.

| Ingredient | Amount, g |
|---|---|
| Coated Magnesium sulfate light green beads | 2.4 |
| Coated Potassium sulfate light blue beads | 4.70 |
| Coated Sodium sulfate green beads | 26.25 |
| Sorbitol | 20 g |
| Gelling agent | 0.5 g |
| Flavoring agent | 0.25 |

Coated bead of three salts listed in Table 42 were prepared using the method described before. The beads were color coded. The beads were mixed with sorbitol, gelling agent and a flavoring agent. The mixture was filled in a pouch. The mixture is added to 500 mL water and stir well. It forms a thick gel. The gel contains suspended salt beads. The gel is consumed. The contained is rinsed with water, which is then consumed.

Table 43 lists the colonoscopy formulation composition of batch 65

TABLE 43
Colonoscopy composition batch #65

| Ingredient | Amount, g |
|---|---|
| PEG 3350 | 210 |
| Coated sodium sulfate green beads | 11.25 |
| Coated Sodium chloride blue beads | 4.05 |
| Coated Potassium chloride yellow beads | 2.22 |
| Ascorbic acid | 4.7 |
| Sodium ascorbate | 5.9 |
| Bisacodyl enteric coated tablet, 10 mg | One |

Pouch 1 contains PEG 3350, ascorbic acid, and sodium ascorbate. Pouch 2 contains three salts and one biscodyl enteric coated tablet. Contents of pouch 1 are dissolved in 500 mL water. Contents of pouch 2 are swallowed using the solution of PEG 3350 followed by water. The second dose is taken on the day of colonoscopy.

It is possible that patient would place the granules in the mouth in small amounts and drink the gel. In that case, even formulation #60 will be acceptable.

### Example 30

### Scale-Up Development of Enteric-Coated Electrolyte Pellets Using Pure Sugar Beads

The objective of this development was to formulate electrolyte-loaded pellets with electrolyte concentrations approximately 1.5-1.6 times higher than conventional Golytely formulations and approximately 1.2-1.3 times higher than conventional Suprep formulations, using all water-soluble excipients to ensure no insoluble residue remains in the GI tract during colonoscopy. The manufacturing process consisted of four stages: (1) preparation of pure sugar pellets using a tangential spray conical rotor processor (APCG 175); (2) electrolyte loading onto sugar pellets using the same tangential spray conical rotor; (3) application of a seal coat (HPMC E5) using bottom spray in a fluid bed processor; and (4) application of an enteric coat (Eudragit L30D-55 or HPMC Phthalate) using bottom spray in a fluid bed processor.

Golytely-Type Formulation (EL-2602): Pure sugar pellets (18-40 mesh starter sugar spheres, 24.04% of formulation) were prepared as described above. The electrolyte loading powder comprised sodium sulfate anhydrous (40.74%), sodium bicarbonate (12.13%), sodium chloride (10.51%), potassium chloride (5.36%), and extra loading powder (0.70%). PVP K30 (6.72%) was used as the binder in a 50% aqueous solution. The electrolyte mixture was loaded onto the sugar pellets by tangential spray in the conical rotor processor. The resulting Golytely-type formulation achieved an electrolyte ratio of approximately 1.45-1.57 times that of commercial Golytely, enabling a potential dosage reduction of approximately 35-40% (100 g of pellets instead of 150 g of Golytely).

The manufacturing equipment for the Golytely-type formulation comprised, in sequence: Stage 1 (pure sugar pellets)—a pulverizer for milling pure sugar loading powder, a 12-inch sifter with 150-mesh sieve, a balance (0.020-10.0 kg capacity), an overhead mechanical stirrer for sugar syrup binder preparation, and an APCG-175 tangential conical rotor with tangential powder loader for powder loading onto starter sugar crystals (60-80 mesh); Stage 2 (electrolyte loading)—a pulverizer for milling electrolyte ingredients, a 12-inch sifter with 150-mesh sieve, a balance, a 5 kg contablender for mixing loading powder, an overhead mechanical stirrer for binder solution preparation, the APCG-175 tangential conical rotor for electrolyte loading by tangential spray, and drying within the tangential rotor granulator to bring moisture below 2%, followed by sifting through 10, 12, 14, 16, 18, and 20 mesh sieves; Stage 3 (seal coating)—a balance, an overhead mechanical stirrer with hot plate for seal coat solution preparation, the APCG-175 in bottom spray configuration for seal coating with HPMC E5, drying to bring moisture below 2%, and sifting through 10, 12, 14, 16, and 18 mesh sieves; and Stage 4 (enteric coating)—a balance, an overhead mechanical stirrer for enteric coat solution preparation, the APCG-175 in bottom spray configuration for enteric coating, drying until moisture is below 2%, an analytical moisture balance for IPQC, sifting through 8, 10, and 20 mesh sieves, and packing in aluminum pouches.

The seal coat for the Golytely-type formulation comprised HPMC E5 (3.20%) and PEG 6000 (0.32%) applied from a 10% w/w aqueous solution by bottom spray. The enteric coat was applied in two variants: (A) Eudragit L30D-55 at 20% coating level (as dry polymer) with triethyl citrate as plasticizer in purified water; and (B) HPMC Phthalate at 15% coating level with triethyl citrate as plasticizer in IPA/acetone solvent system.

Test results for the Golytely-type formulation at each stage were as follows. Stage 2 (electrolyte-loaded pellets): moisture content (LOD) 1.64%; solubility in purified water 1.15 minutes with clear solution; particle size distribution 14-30 mesh=88.42%; assay by AAS: total sodium 116%, potassium 88.9%; dissolution in water 100% within 10 minutes. Stage 3 (seal-coated pellets): LOD 1.27%; solubility 3.49 minutes with clear solution; particle size distribution 14-25 mesh=99.97%; pellets remained intact longer (up to 10 minutes) in dissolution media but produced clear solution. Stage 4A (Eudragit L30D-55, 20% coating): LOD 0.80%; particle size distribution 12-25 mesh=99%; pellets remained intact after 30 minutes in acidic conditions. Dissolution: in pH 1.2 for 30 minutes=0.5% (no release in acid); in pH 7.2 for 30 minutes=86.2%; in pH 7.2 for 60 minutes=105.6% (complete release). Potassium dissolution: in pH 1.2 for 30 minutes=1.76%; in pH 7.2 for 15 minutes=71.13%; in pH 7.2 for 30 minutes=121.18% (complete release). Assay by AAS: total sodium 86.92%, potassium 94.17%.

Stage 4B (HPMC Phthalate, 15% coating): LOD 1.45%; particle size distribution 12-25 mesh=99%; pellets remained intact after 30 minutes in acidic conditions. Dissolution for total sodium: in pH 1.2 for 30 minutes=1.525% (no release in acid); in pH 7.2 for 15 minutes=73.95%. Dissolution for potassium: in pH 1.2 for 30 minutes=2.55% (no release in acid); in pH 7.2 for 15 minutes=70.43%; in pH 7.2 for 30 minutes=107.8% (complete release). Assay by AAS: total sodium 87.89%, potassium 91.27%. Notably, HPMC Phthalate at 15% achieved comparable acid resistance to Eudragit at 20%, and the release at 15 minutes in pH 7.2 was slightly reduced compared to Eudragit, indicating that the HPMC Phthalate coating percentage can potentially be reduced further to achieve faster dissolution while maintaining acid protection.

Suprep-Type Formulation (ST-2601): Pure sugar pellets (16-25 mesh starter sugar spheres, 20.00% of formulation) were prepared using larger starter crystals to produce smoother spherical pellets with final particle size in the 16-25 mesh range. The electrolyte loading powder comprised sodium sulfate anhydrous (46.48%), potassium sulfate (8.31%), magnesium sulfate heptahydrate (4.25%), citric acid (0.80%), and malic acid (0.80%). Sugar syrup (23.08%) was used as the binder for the electrolyte loading step. The electrolyte mixture was loaded onto the sugar pellets by tangential spray in the conical rotor processor. The Suprep-type formulation achieved an electrolyte concentration of approximately 1.2 times that of commercial Suprep, with further optimization expected to reach 1.4-1.5 times.

The seal coat for the Suprep-type formulation comprised HPMC E5 (3.14%) and PEG 6000 (0.31%) applied from a 10% w/w aqueous solution by bottom spray. Three enteric coating variants were evaluated: (A) Eudragit L30D-55 at 5% coating level; (B) HPMC Phthalate at 12% coating level; and (C) HPMC Phthalate at 5% coating level.

Test results for the Suprep-type formulation were as follows. Stage 2 (electrolyte-loaded pellets): LOD 1.64%; solubility in purified water 1.15 minutes with clear solution; particle size distribution 14-30 mesh=88.42%; assay by AAS: sodium 87.89%, potassium 92.5%, magnesium 72.25%; dissolution in water 100% within 10 minutes. Stage 3 (seal-coated pellets): LOD 2.95%; solubility 3.18 minutes with clear solution; particle size distribution 10-14 mesh=98%. Stage 4A (Eudragit L30D-55, 5% coating): LOD 1.42%; particle size distribution 10-20 mesh=99%; assay: sodium 84.87%, potassium 90.02%, magnesium 70.01%. Dissolution for sodium: pH 1.2 for 30 minutes=0.3% (no release in acid); pH 7.2 for 15 minutes=105% (complete release). Dissolution for potassium: pH 1.2 for 30 minutes=0.36%; pH 7.2 for 15 minutes=greater than 100%. Dissolution for magnesium: pH 1.2 for 30 minutes=3.61%; pH 7.2 for 15 minutes=94.67%.

Stage 4B (HPMC Phthalate, 12% coating): LOD 1.36%; particle size distribution 10-14 mesh=97%; assay: sodium 85.3%, potassium 89.5%, magnesium 58.16%. Dissolution for sodium: pH 1.2 for 30 minutes=1.01% (no release in acid); pH 7.2 for 15 minutes=110.25%. Dissolution for potassium: pH 1.2 for 30 minutes=0.21%; pH 7.2 for 15 minutes=162% (interference due to potassium phosphate in buffer). Dissolution for magnesium: pH 1.2 for 30 minutes=4.66%; pH 7.2 for 15 minutes=122%. At the 12% HPMC Phthalate level, the pellets formed a sticky bulky structure in solubility testing, indicating this coating percentage may be at or above the upper practical limit.

Stage 4C (HPMC Phthalate, 5% coating): LOD 1.23%; particle size distribution 10-14 mesh=96%; assay: sodium 72.5%, potassium 90.93%, magnesium 54.49%. Dissolution for sodium: pH 1.2 for 30 minutes=2.07% (no release in acid); pH 7.2 for 15 minutes=117.55%. Dissolution for potassium: pH 1.2 for 30 minutes=0.5%; pH 7.2 for 15 minutes=130% (interference due to potassium phosphate in buffer). Dissolution for magnesium: pH 1.2 for 30 minutes=4.01%; pH 7.2 for 15 minutes=120.34%. Notably, HPMC Phthalate at only 5% coating achieved excellent acid resistance (less than about 2.07% release at pH 1.2) while providing complete release at intestinal pH within 15 minutes.

The dissolution values for potassium exceeding 100% in certain tests were attributed to interference from potassium phosphate used in the dissolution buffer medium, requiring further analytical method validation. Assay values below 100% (sodium approximately 85-88%, potassium approximately 89-94%, magnesium approximately 54-72%) were attributed to loss of loading powder during the electrolyte loading stage in the fluid bed processor. This can be addressed in further batch optimization through adequate overages or by appropriate process controls. The total sugar content in the formulations was approximately 22-24% for the Golytely-type formulation and approximately 38-42% for the Suprep-type formulation.

Both Eudragit L30D-55 and HPMC Phthalate coatings effectively protected electrolyte cores in acidic conditions while permitting full release at intestinal pH. The enteric coating also masks the taste of electrolytes, enabling the pellets to be swallowed with water or with a laxative solution without the patient tasting the electrolytes during transit through the mouth. As a result, the volume of water needed to administer electrolytes is decreased substantially compared to conventional colonoscopy preparations. The electrolyte concentration achieved was approximately 1.5-1.6 times higher than conventional Golytely compositions and approximately 1.2-1.4 times higher than conventional Suprep compositions, potentially reducing patient dosage intake by approximately 35-40% for both formulation types. The Golytely-type developed product contains approximately 1.5 times the electrolyte concentration of Golytely, with the following comparative amounts per dose: sodium sulfate anhydrous 31.07 g (Eudragit variant) and 33.78 g (HPMC Phthalate variant) versus 21.5 g in Golytely; sodium bicarbonate 9.25 g and 10.06 g versus 6.37 g; sodium chloride 8.01 g and 8.71 g versus 5.53 g; and potassium chloride 4.09 g and 4.45 g versus 2.82 g. The Suprep-type developed products (ST-2601A, ST-2601B, and ST-2601C) contain approximately 1.2 times the electrolyte concentration of Suprep, with the following comparative amounts: sodium sulfate anhydrous 42.58 g, 39.69 g, and 42.36 g versus 35 g in Suprep; potassium sulfate 7.62 g, 7.10 g, and 7.58 g versus 6.26 g; and magnesium sulfate heptahydrate 3.89 g, 3.63 g, and 3.87 g versus 3.2 g

### Example 31

### Analytical Methods for Coated Electrolyte Pellets

Atomic Absorption Spectroscopy (AAS) was used for percent assay and dissolution studies of the coated electrolyte pellets. AAS was found suitable for determining cation concentrations (sodium, potassium, magnesium) in dissolution media samples. The dissolution studies were conducted at pH 1.2 (simulated gastric fluid) for 30 minutes and at pH 7.2 (simulated intestinal fluid) for 15, 30, and 60 minutes. Moisture content was determined by loss on drying (LOD) at 105° C. for 5 minutes. Solubility was tested by placing 2 g of pellets in 50 g purified water and measuring time to complete dissolution. In some embodiments, the analytical method for determining the dissolution profile of the coated inorganic salt(s) is selected from ICP-MS, Atomic Absorption Spectroscopy (AAS), ion chromatography, and any combination thereof. It was observed that use of potassium phosphate buffer to prepare the dissolution medium resulted in interference with potassium measurements, producing artificially high values. Alternative buffer systems or analytical methods that can independently analyze anions as well as cations are preferred for validated dissolution studies.

### Examples 32-41

The following Examples focuses on non-colonoscopy compositions, which includes but is not limited to sports drinks, formulations containing a mixture of various key salts to patients suffering from diarrhea, vomiting etc., formulations with specific single electrolytes such as sodium chloride, potassium chloride or sodium bicarbonate. In colonoscopy compositions, all the formulations must have only water-soluble components including polymers used in the coating of granules, spheres etc. In the non-colonoscopy composition, there is no such requirement and water-insoluble polymers such ethyl cellulose can be used.

### Example 32

In the trial run, ethyl cellulose coated granules-200 mg of sodium bicarbonate, 100 mg sodium chloride and 50 mg potassium chloride were added to 20.03 g of a sports drink. The granules settled down but could be resuspended with shaking. The pH of solution was 7.73.

### Batch 1: Gel Formulation

TABLE 44
Composition of a gel formulation

| Ingredient | Amount in 36 g |
|---|---|
| Blanose CMC 7H4CF | 3.58 g |
| Sorbitol | 32.41 g |

Both components were mixed in a Ziplock bag. Added 4 g of mixture to 100 mL water. It formed a good gel.

### Batch 2: Gel Formulations

TABLE 45
Composition of gel formulation batch 2

| Ingredient | Batch # used | Amount |
|---|---|---|
| Blend of Blanose + Sorbitol | 1 | 8.0616 |
| Coated Potassium chloride | 22 | 171.2 mg |
| Coated Sodium bicarbonate | 26 | 254.2 mg |
| Coated Sodium chloride | 25 | 285.8 mg |

All the powders in a cup and with spatula were weighed. 100 mL water was added. After the salt particles settled down, there was no salty taste after 0 time, after 3 hours and after overnight storage.

There was some sweet and a slight polymeric taste.

### Batch 3: Suspension in Water as a Control

TABLE 46
Composition of formulation batch 3

| Ingredient | Batch # used | Amount |
|---|---|---|
| Potassium chloride | 22 | 171.1 mg |
| Sodium bicarbonate | 26 | 254.5 mg |
| Sodium chloride | 25 | 285.5 mg |

The coated granules of salts were suspended in 100 mL water. There was no saltiness.

TABLE 47
Density values of various coated electrolytes

| | | | | Tap |
|---|---|---|---|---|
| | Electrolyte, | | Density, | Density, |
| Batch# | color | Description | g/mL | g/mL |
| 16 | KCl, yellow | Original granules, | 0.929 | 0.978 |
| | | smaller, Enteric coated | | |
| 17 | Na2SO4, | Small granules with | 0.749 | 0.842 |
| | Green | HPC, Enteric coated | | |
| 20 | NaHCO3, | Granules with HPC, | 0.654 | 0.696 |
| | White | Enteric coated | | |
| 21 | NaCl, Blue | Granules with HPC, | 0.996 | 1.048 |
| | | EC coated | | |
| 22 | KCI, | Granules with HPC, | 0.821 | 0.864 |
| | Yellow | EC coated | | |
| 23 | Na2SO4, | Granules with HPC, | 1.020 | 1.074 |
| | Green | EC coated | | |
| 24 | NaHCO3, | Smaller granules | 0.732 | 0.787 |
| | White | coated with EC | | |
| 25 | NaCl, Blue | Larger granules | 0.809 | 0.852 |
| | | coated with EC | | |
| 26 | NaHCO3, | Granules coated with | 0.691 | 0.736 |
| | | HPMC, Enteric, and | | |
| | White | PEG Stearate | | |

The density gets affected by various factors. Overall, the density is about 0.82 g/mL. Tap density (tapped for 20-25 time) was always slightly higher than the as is density.

### Example 33

EC coated KCl coated granules in 000 capsules.

The volume of 000 capsule is 1.37 mL. The density of KCl granules was observed to be 0.929 g/mL and 0.821 g/mL. Thus, one can fill 1.27 g and 1.12 g granules in one capsule, respectively. The amount of KCl in each capsule will be 0.699 g and 0.617 g, respectively. Average human beings cannot ingest this capsule. Thus, it is recommended to open the capsule and transfer the contents in a spoon or in a small glass. The granules can be administered with water. The granules were tasteless and there was no need to add any excipients. For higher doses, more than one capsule can be used. The granules can be sprinkled over edible semisolids, such as Jello, Jelly, Apple sauce etc.

### Example 34

EC coated KCl granules in a stick-packs or pouches.

About 3 grams of coated KCl granules were filled in a stick-pack or in a pouch. The primary packaging devices were opened, and the contents transferred into a spoon or a small cup. The granules were be administered with water. The granules were tasteless and there was no need to add any excipients. The granules can be sprinkled over edible semisolids, such as Jello, Jelly, Apple sauce etc.

### Example 35

EC coated NaCl granules in 000 capsules or in stick-packs or pouches.

The density values of EC-coated NaCl granules were observed to be 0.996 g/mL and 0.809 g/mL for small and large granules. Thus, 1.36 g and 1.11 g granules can fill one capsule. Pouches or stick-packs can be filled with 3-4-gram granules. The granules can be administered “as is” or it can be sprinkled over edible semi-solids, such as Jello, Jelly, Apple sauce etc.

### Example 36

### EC-Coated Electrolyte Granules in a Stick-Pack or in a Pouch

The coated electrolytes tend to sink when added to water. However, if these electrolytes are added to a gel, the electrolytes remain suspended. Table 48 lists the composition of a formulation, which forms a gel when added to water.

TABLE 48
Gel forming composition of electrolytes.

| Ingredient | Amount, g |
|---|---|
| NaCl granules | 3 |
| KCl granules | 2 |
| NaHCO3 granules | 2 |
| Sorbitol | 14 |
| Sodium CMC | 0.5 |
| Xanthan gum | 0.5 |
| Peppermint flavor | 0.2 |
| Acesulfame | 0.2 |

### Example 37

### Orally Disintegrating Tablets

Orally disintegrating tablets can be orally administered allowing them to disintegrate. Some elderly people with dry mouth issue do not like this approach as the tablet may further dry their mouth.

The ODT can be administered by presoaking it with water in a spoon. The tablet was placed in a spoon and water was added. The tablet absorbed water immediately, resulting in disintegration and swelling of the tablet. This soft mass can be eaten easily by children or elderly patients. Table 49 lists a typical composition of the ODT product. The composition can be easily modified to meet new requirements, if any.

TABLE 49
Orally disintegrating tablet formulation

| Ingredient | Percent |
|---|---|
| Sodium chloride coated granules | 24 |
| Sorbitol | 40 |
| Sodium starch glycolate | 20 |
| Microcrystalline cellulose | 15 |
| Acesulfame potassium | 0.5 |
| Magnesium stearate | 0.5 |

### Example 38

Effervescent disintegrating tablets in a spoon.

All effervescent products are normally preferred by patients. Table 50 lists the composition of an effervescent tablet to be placed in a spoon. The effervescent tablet was placed in a spoon and water is added. Tablet disintegrated producing some fuzz. The effervescence helped disintegration of tablets and it disintegrated in a short time.

TABLE 50
Composition of effervescent disintegrating tablets in spoon.

| Ingredient | Amount, g |
|---|---|
| Sodium chloride granules | 2 |
| Potassium chloride granules | 1 |
| Sodium Starch Glycolate | 0.75 |
| Microcrystalline cellulose | 0.5 |
| Acesulfame K | 0.2 |
| Magnesium Stearate | 0.2 |
| Milled citric acid monohydrate | 0.75 |
| Sodium Bicarbonate | 1.2 |
| Ludiflash | 2 |

### Example 39

### Effervescent Disintegrating Tablets in a Glass of Water

A large volume of soft swollen mass in a spoon. Sometimes, patients prefer to drink water with fuzz. Here, it is possible to administer a mixture with large amount of salts.

TABLE 51
Composition of effervescent disintegrating tablets in a glass of water

| Ingredient | Amount, g |
|---|---|
| Sodium chloride granules | 3 |
| Potassium chloride granules | 2 |
| Sodium Starch Glycolate | 0.75 |
| Microcrystalline cellulose | 0.5 |
| Acesulfame K | 0.2 |
| Magnesium Stearate | 0.2 |
| Milled citric acid monohydrate | 1 |
| Sodium Bicarbonate | 1.5 |
| Ludiflash | 2.5 |

### Example 40

### A Gelling Tablet

For geriatric patients swallowing big bulky tablet is very hard.

TABLE 52
Composition of a gelling tablets with coated electrolytes

| Ingredient | Amount, g | |
|---|---|---|
| NaCl granules | 1.5 | |
| KCl granules | 1.5 | |
| Sodium starch glycolate | 1 | g |
| Kollidon 90 F | 0.5 | |
| Microcrystalline cellulose | 1.3 | g |
| Xanthan gum | 100 | mg |
| Acesulfame K | 50 | mg |
| Magnesium Stearate | 50 | mg |

The gelling tablet is placed in a tablespoon and water is added. The tablet absorbs water and starts swelling. Add sufficient water so that the tablet swells fully and forms a gel. The soft mass is eaten like an apple sauce. This is good mainly for elderly patients or kids.

### Example 41

### “On the Run Six Pack” Powder Mixture

Athletes or long-distance runners sweat a lot. It is commonly known that electrolytes are lost in the sweat and it can affect the performance. Thus, it is important to replenish electrolytes during the activity. The “On the Run Six-Pack” contains three essential electrolytes and three vitamins. Table 53 lists the composition of this formulations. It also contains a gelling agent, a sweetener and a flavor of one's choice. The key aspect is formation of a gel by just mixing with 12-16 Oz of water in a “trainer's bottle”.

TABLE 53
Composition of “On the Run Six Pack” powder mixture.

| Ingredients | Amount | |
|---|---|---|
| Sodium chloride granules | 3 | g |
| Potassium chloride granules | 1 | g |
| Calcium sulfate granules | 1 | g |
| Vitamin C | 500 | mg |
| Vitamin B12 | 10000 | mcg |
| Thiamin B1 | 200 | mg |
| Sorbitol | 5 | g |
| Xanthan gum | 0.25 | g |
| Flavoring agent, Orange | 100 | mg |
| Sweetener, Acesulfame | 100 | mg |

A thick solution of xanthan gum in water was added to a mixture containing sorbitol and the sweetener. The wet mass was passed through a 14-mesh sieve and dried at 40° C. Vitamin C, vitamin B12, thiamin and the flavoring agent were mixed with the granules one by one with continuous mixing. The granules of three electrolytes were mixed with this mass. The powder when shaken with 12-16 of water in a bottle, formed a gel with a satisfactory thickness. The bottle is shaken before drinking the gel. The gel can be consumed during the run or game.

It should be noted that the above-mentioned preferred examples are further, non-limiting, and detailed descriptions of the embodiments of the present invention and are only for illustrating the technical concepts and features of the present invention. They are provided for better understanding and implementation of the present invention by those skilled in the art, rather than limiting the scope of the present invention. All equivalent changes and modifications made according to the spirit of the present invention should fall within the protection scope of the present invention.

What is claimed is:

1. A coated inorganic salt comprising:
(a) one or more inorganic salt;
(b) a core comprising the one or more inorganic salt; and
(c) a first polymer coating layer enclosing the core; wherein the first polymer coating is an enteric or a reverse enteric coating, wherein the enteric coating dissolves in the small intestine and the reverse enteric coating dissolves in the stomach, wherein the inorganic salt ranges from about 5% to about 95% by weight in the coated inorganic salt.

2. The coated inorganic salt of claim 1, wherein the one or more inorganic salt is selected from the group consisting of sodium salt, potassium salt, calcium salt, iron salt, copper salt, zinc salt, manganese salt, magnesium salt, molybdenum salt, cobalt salt, and chromium salt, and/or the inorganic salt comprise an anion selected from the group consisting of chloride, fluoride, sulfate, bisulfate, carbonate, bicarbonate, phosphate, and citrate.

3. The coated inorganic salt of claim 1, further comprising an undercoat between the core and the first polymer coating layer, wherein the undercoat prevents interaction between the inorganic salt and the first polymer coating layer.

4. The coated inorganic salt of claim 1, further comprising a second polymer coating enclosing the core and the first polymer coating, wherein the second polymer coating delays the release of the inorganic salt, and wherein the second polymer comprises wax, PEG stearate coating, and any combination thereof.

5. The coated inorganic salt of claim 1, wherein the core further comprises an inner core that consists essentially of one or more soluble materials, wherein the one or more inorganic salt are coated onto the inner core.

6. The coated inorganic salt of claim 5, wherein the soluble materials are water-soluble sugar crystals.

7. The coated inorganic salt of claim 6, wherein the one or more water-soluble sugar crystals are selected from maltose, sucrose, glucose, fructose, lactose, and combinations thereof.

8. The coated inorganic salt of claim 5, wherein all components of the coated inorganic salt are water soluble such that no insoluble residue remains after dissolution.

9. The coated inorganic salt of claim 1, wherein the enteric coating comprises hydroxypropyl methylcellulose phthalate (HPMC Phthalate), a methacrylic acid copolymer, or a combination thereof.

10. The coated inorganic salt of claim 1, further comprising a layer for providing extended release of the inorganic salt.

11. The coated inorganic salt of claim 1, wherein the coated inorganic salt is in the form of a bead.

12. A dosage form comprising the coated inorganic salt of claim 1.

13. The dosage form of claim 12, wherein the dosage form is selected from the group consisting of bead, multiple particulates, tablet, capsule, caplet, sachet, granule, and liquid suspension.

14. The dosage form of claim 12, which is a bead or multiple particulates.

15. The dosage form of claim 12, which is a hard or soft gelatin capsule.

16. The dosage form of claim 12, wherein the inorganic salt is in an effective amount to provide a normal electrolyte level.

17. A method of providing coated inorganic salt to a subject in need thereof, the method comprising administering to the subject the coated inorganic salt of claim 1.

18. The method of claim 17, wherein the subject is electrolyte deficient.

19. The method of claim 17, wherein the method reduces the taste of the salt.

20. The method of claim 17, wherein the coated inorganic salt is delivered to a subject prior to a colonoscopy procedure.
